(19) United States
(12) Patent Application Publication
(10) Pub. No.: US 2022/0344031 A1
(43) Pub. Date: Oct. 27, 2022

(54) METHODS FOR TRANSFERRING A PLURALITY OF MEDICAL IMAGES

(71) Applicant: Siemens Healthcare GmbH, Erlangen (DE)

(72) Inventor: Christian KAETHNER, Forchheim (DE)

(73) Assignee: Siemens Healthcare GmbH, Erlangen (DE)

(21) Appl. No.: 17/723,896

(22) Filed: Apr. 19, 2022

(30) Foreign Application Priority Data
Apr. 22, 2021 (DE) 10 2021 204 020.9

(51) Int. Cl. G16H 30/20; G16H 50/20

(52) U.S. Cl. CPC G16H 30/20; G16H 50/20

(57) ABSTRACT

At least one example embodiment relates to a computer-implemented method comprising receiving a plurality of medical images with a transmitting apparatus interface, determining a first base image, based on a first medical image, with a transmitting apparatus computing unit, transferring the first base image, determining a first change information item, based on the first medical image and a second medical image, with the transmitting apparatus computing unit, determining a first modified base image based on the first change information item and the first base image, checking at least one of the second medical image or the first modified base image, transferring the second medical image with the transmitting apparatus interface to the receiving apparatus interface based on the result of the checking.

### CROSS-REFERENCE TO RELATED APPLICATION(S)

The present application hereby claims priority under 35 U.S.C. § 119 to German patent application number DE 102021204020.9 filed Apr. 22, 2021, the entire contents of which are hereby incorporated herein by reference.

### FIELD

At least some example embodiments relate to computer-implemented methods for transferring a plurality of medical images from a transmitting apparatus to a receiving apparatus. Example embodiments also relate to transmitting methods, receiving methods, transmitting apparatuses, receiving apparatuses, transfer systems, computer program products and computer-readable storage mediums.

### BACKGROUND

It is known to carry out an image-assisted medical intervention in the medical field. Therein, a plurality of medical images are displayed to a medical practitioner who carries out the medical intervention on a patient on the basis of the plurality of medical images. Alternatively or additionally, the medical practitioner can generate a diagnosis for the patient on the basis of the plurality of medical images. The plurality of medical images can thus be of decisive importance for a decision-making process by the medical practitioner. The plurality of medical images therein comprises a mapping of at least one portion of the patient that is relevant for the decision-making process. The plurality of medical images is therein typically acquired with a medical technology device. The medical technology device can be, for example, an X-ray device, a computed tomography (CT) device, a C-arm, a magnetic resonance tomography (MRT) device, an ultrasound device, an angiography system, an electrocardiography system, a positron emission tomography (PET) system and/or a single photon emission computed tomography (SPECT) system. The plurality of medical images is therein transferred by the medical technology device to a receiving apparatus. Alternatively, the plurality of medical images can initially be, for example, processed and subsequently transferred to the receiving apparatus. In other words, the plurality of medical images can be transferred by a transmitting apparatus to a receiving apparatus. The receiving apparatus can then provide the plurality of medical images to the medical practitioner.

The receiving apparatus can therein be arranged spatially separated from the transmitting apparatus. The receiving apparatus can be arranged, for example, in the same room as the transmitting apparatus or in a different room or in a different building or in a different city or in a different country. The medical practitioner can carry out the image-assisted medical intervention “remotely” on the basis of the plurality of medical images provided by the receiving apparatus.

It is known that medical images comprise a large quantity of data. This can lead, in particular, with a limited bandwidth to a time delay due to the transfer of the plurality of medical images from the transmitting apparatus to the receiving apparatus. In an imaging-assisted medical intervention, in particular, it is necessary however that the medical images are transferred to the receiving apparatus with a minimum time delay. It is desired that the plurality of medical images are transferred error-free.

From the field of video conferencing, in order to reduce the transferred data, it is known to transfer initially just one base image (keyframe). In images temporally following the base image, only a change information item (keypoints) relating to the base image is transferred. The receiving apparatus then determines, by means of a neural network, the images following the base image based upon the base image and the change information item. A method of this type has been developed, for example, by NVIDIA (https://www.youtube.com/watch?v=NqmMnjJ6GEg).

### SUMMARY

In a medical application however, for example, for carrying out an image-assisted medical intervention, the safety standards relating to image quality during transfer of the plurality of medical images is substantially larger than for a video conference. For example, the safety standards specify that it is ensured that all the medically relevant details are transferred in the medical images. In particular, no anomaly in a medical image should be “lost” in the transfer, or not be transferred. In other words, it should be ensured that a transferred image corresponds to the original image.

At least one example embodiment provides a method which enables an error-free and data-reducing transfer of a plurality of medical images.

This may be achieved by way of a method for transferring a plurality of medical images from a transmitting apparatus to a receiving apparatus, by way of a transmitting method, by way of a receiving method, by way of a transmitting apparatus, by way of a receiving apparatus, by way of a transfer system, by way of a computer program product and by way of a computer-readable storage medium according to the independent claims. Advantageous developments are disclosed in the dependent claims and in the following description.

At least one example embodiment provides a computer-implemented method for transferring a plurality of medical images by a transmitting apparatus comprising a transmitting apparatus interface and a transmitting apparatus computing unit to a receiving apparatus comprising a receiving apparatus interface and a receiving apparatus computing unit, wherein the plurality of medical images comprises a temporal sequence, wherein the plurality of medical images comprises at least one first medical image and one second medical image, wherein the first medical image has been captured temporally before the second medical image, wherein the method comprises receiving the plurality of medical images with the transmitting apparatus interface; determining a first base image, dependent upon the first medical image, with the transmitting apparatus computing unit; transferring the first base image with the transmitting apparatus interface to the receiving apparatus interface; determining a first change information item, based on the first and second medical images, with the transmitting apparatus computing unit; determining a first modified base image based on the first change information item and the first base image; checking at least one of the second medical image or the first modified base image; and transferring the second medical image with the transmitting apparatus interface to the receiving apparatus interface based on the result of the checking of the at least one of the second medical image or the first modified base image.

### BRIEF DESCRIPTION OF THE DRAWINGS

The above described properties, features and advantages of example embodiments become more clear and comprehensible in conjunction with the following figures and the descriptions thereof. The figures and descriptions are not intended to restrict example embodiments in any way.

In the different figures, the same components are provided with corresponding reference signs. The figures are in general not to scale.

In the figures:

FIG. 1 shows a first exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 2 shows a second exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 3 shows a third exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 4 shows a fourth exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 5 shows a fifth exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 6 shows an exemplary embodiment of a method step of checking a second medical image and/or a first modified base image,

FIG. 7 shows a sixth exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 8 shows a seventh exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 9 shows an eighth exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 10 shows a ninth exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 11 shows a tenth exemplary embodiment of a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit,

FIG. 12 shows a transmitting apparatus,

FIG. 13 shows a receiving apparatus, and

FIG. 14 shows a transfer system.

### DETAILED DESCRIPTION

An inventive achievement of the object is described below, both in relation to the claimed devices and also in relation to the claimed method. Features, advantages or alternative embodiments mentioned herein are also transferable similarly to the other claimed subject matter and vice versa. In other words, the object-related claims (which are directed, for example, to a device) can also be further developed with the features disclosed or claimed in relation to a method. The corresponding functional features of the method are thereby embodied by corresponding physical modules.

At least one example embodiment relates to a computer-implemented method for transferring a plurality of medical images from a transmitting apparatus comprising a transmitting apparatus interface and a transmitting apparatus computing unit to a receiving apparatus comprising a receiving apparatus interface and a receiving apparatus computing unit. The plurality of medical images therein comprises a temporal sequence. The plurality of medical images comprises at least one first medical image and one second medical image. Therein, the first medical image has been determined temporally before the second medical image. The method comprises a method step of receiving the plurality of medical images with the transmitting apparatus interface. The method further comprises a method step of determining a first base image dependent upon the first medical image with the transmitting apparatus computing unit. The method further comprises a method step of transferring the first base image with the transmitting apparatus interface to the receiving apparatus interface. The method further comprises a method step of determining a first change information item dependent upon the first and the second medical image with the transmitting apparatus computing unit. The method further comprises a method step of determining a first modified base image dependent upon the first change information item and the first base image. The method further comprises a method step of checking the second medical image and/or the first modified base image. The method further comprises a method step of transferring the second medical image with the transmitting apparatus interface to the receiving apparatus interface dependent upon the result of the checking of the second medical image and/or the first medical base image.

The plurality of medical images can therein be captured with a medical technology device. The medical technology device can be, for example, an X-ray device, a computed tomography (CT) device, a C-arm, a magnetic resonance tomography (MRT) device, an ultrasound device, an angiography system, an electrocardiography system, a positron emission tomography (PET) system and/or a single photon emission computed tomography (SPECT) system. The plurality of medical images can therein be acquired during a medical intervention and/or operation on a patient, in particular. The plurality of medical images can therein map at least a portion or region of the patient. In other words, every medical image of the plurality of medical images can comprise a mapping of the at least one portion or region of the patient. In particular, every medical image comprises a relevant region and/or portion for the medical intervention. The plurality of medical images thus serves as the basis for an image-assisted medical intervention. The plurality of medical images can serve, in particular, for a medical practitioner who carries out the image-assisted medical intervention, as the basis for a decision in relation to the image-assisted medical intervention. Alternatively, the plurality of medical images can serve, independently of a medical intervention, as a decision-making basis for the medical practitioner. For example, the medical practitioner can generate a diagnosis for the patient on the basis of the plurality of medical images. Therein, the patient can be a human. Alternatively, the patient can be an animal or an object.

The plurality of medical images therein comprises a temporal sequence of medical images. In other words, the medical images of the plurality of medical images can be captured in a temporal sequence. In particular, the medical images of the plurality of medical images can thus be arranged temporally.

The plurality of medical images therein comprises at least one first and at least one second medical image. Therein, the first medical image has been captured temporally before the second medical image.

The plurality of medical images can, in particular, be pre-processed. For example, an image processing algorithm for noise suppression and/or for edge enhancement and/or for contrast correction and/or for segmentation, etc. can be used in advance. In particular, the plurality of medical images can be already compressed. For example, the plurality of medical images can be compressed by means of a binning process. Compressed means that the data quantity included by the plurality of medical images is reduced in comparison with the initial and/or original medical images.

In embodiments, the medical images of the plurality of medical images can be reconstructed from raw data. The raw data is therein captured with the medical technology device.

The transmitting apparatus can be, in particular, the medical technology device. Alternatively, the transmitting apparatus can be an apparatus which pre-processes the plurality of medical images. The pre-processing can comprise, for example, a reconstruction of the medical images from raw data and/or an image processing and/or an image compression. The transmitting apparatus comprises a transmitting apparatus interface and a transmitting apparatus computing unit. The transmitting apparatus interface can comprise a plurality of individual sub-transmitting apparatus interfaces. The transmitting apparatus computing unit can therein comprise a plurality of individual sub-transmitting apparatus computing units. The different sub-transmitting apparatus interfaces and/or sub-transmitting apparatus computing units can be configured, in particular, to carry out different method steps carried out by the transmitting apparatus interface and/or the transmitting apparatus computing unit.

The receiving apparatus therein comprises, in particular, a display unit for displaying the transferred medical images. The receiving apparatus can be, in particular, a tablet and/or a personal computer (PC) and/or a laptop computer. The display unit can be, in particular, a screen and/or a monitor. The monitor can be, for example, a field emission display (FED), a liquid crystal display (LCD), a thin film transistor screen (TFT-LCD), a cathode ray tube screen (CRT), a plasma screen and/or a surface conduction electron emitter display (SED). The receiving apparatus comprises a receiving apparatus interface and a receiving apparatus computing unit. The receiving apparatus interface can therein comprise a plurality of individual sub-receiving apparatus interfaces. The receiving apparatus computing unit can therein comprise a plurality of individual sub-transmitting apparatus computing units. The different sub-receiving apparatus interfaces and/or sub-receiving apparatus computing units can be configured, in particular, to carry out different method steps carried out by the receiving apparatus interface and/or the receiving apparatus computing unit.

The receiving apparatus can therein be arranged spatially separated from the transmitting apparatus. For example, the receiving apparatus can be arranged in a different room, in a different building, in a different city, in a different country from the transmitting apparatus.

The expression “transfer of the plurality of medical images” also comprises a transfer of individual image information items of the medical images. In other words, the transfer of the plurality of medical images does not necessarily describe that every medical image of the plurality of medical images is transferred fully. In other words, “transfer of the plurality of medical images” means that all the medically relevant details of the medical image of the plurality of medical images is transferred to the receiving apparatus.

In the method step of receiving the plurality of medical images, the plurality of medical images is received with the transmitting apparatus interface. The receiving of the plurality of medical images can therein comprise the capture of the medical images. Alternatively, the plurality of medical images can be provided by the medical technology device and received by the transmitting apparatus. Alternatively, the plurality of medical images can be provided by a database and received by the transmitting apparatus. The database can be, for example, a picture archiving and communication system (PACS). In particular, the medical images of the plurality of medical images can consequently be received temporally offset and/or in a temporal sequence. For example, the first medical image can be captured temporally before the second medical image.

In the method step of determining a first base image, the first base image is determined dependent upon the first medical image with the transmitting apparatus. Therein, the first base image can be identical to the first medical image. Alternatively, the first base image can be a processed first medical image. In other words, the first base image can correspond to a processed version of the first medical image. In this case, the method step of determining the first base image can comprise a method step of processing the first medical image. The first base image can also be designated a “keyframe”.

The method step of processing the first medical image can comprise, for example, a compression of the first medical image. The compression can comprise, for example, a binning. Alternatively or additionally, the method step of processing the first medical image can comprise, for example, an image processing of the first medical image. The image processing can comprise, for example, a contrast correction and/or a segmentation and/or a use of an edge filter etc.

In the method step of transferring the first base image, the first base image is transferred with the transmitting apparatus interface to the receiving apparatus interface. In other words, the first base image is transferred from the transmitting apparatus to the receiving apparatus. In particular, this can be understood as meaning the same as the expression “the first medical image is transferred”. The method step of transferring the first base image can be carried out by a different sub-transmitting apparatus interface from the method step of receiving the plurality of medical images. In other words, the method steps of transferring the first base image and receiving the plurality of medical images can be carried out by different sub-transmitting apparatus interfaces of the transmitting apparatus interface. Alternatively, the two method steps can be carried out with the same (sub-)transmitting apparatus interface.

In the method step of determining a first change information item, the first change information item is determined dependent upon the first and second medical images with the transmitting apparatus unit. In particular, the first change information item can be determined dependent upon the first base image and the second medical image. The first change information item describes a change between the second medical image and the first medical image. In other words, the first change information item describes what has changed between the first and second medical images. The first change information item can therein comprise a change point and/or a change vector which describes a change of the second medical image as compared with the first medical image. The at least one change point can be designated a “keypoint”.

If the first base image is not equal to the first medical image, the first change information item is determined, in particular, dependent upon the first base image and the second medical image. The first change information item then describes a change between the first base image and the second medical image.

The first change information item can be determined, in particular, by means of a trained function. Therein, the trained function can be applied to the first and second medical images. Therein, the first change information item is generated.

In general, a trained function emulates cognitive functions that link humans to human thinking. In particular, by way of training based upon training data, the trained function can adapt to new circumstances and can recognize and extrapolate patterns.

In general, parameters can be adapted to a trained function by means of training. In particular, for this purpose, a supervised training, a semi-supervised training, an unsupervised training, a reinforcement learning and/or an active learning can be used. Furthermore, representation learning, also known using an alternative expression as “feature learning”, can be used. In particular, the parameters of the trained functions can be adapted iteratively by way of a plurality of training steps.

In particular, a trained function can comprise a neural network, a support vector machine, a random tree or a decision tree and/or a Bayesian network, and/or the trained function can be based upon k-means clustering, Q-learning, genetic algorithms and/or association rules. In particular, a trained function can comprise a combination of a plurality of uncorrelated decision trees and/or an assembly of decision trees (a random forest). In particular, the trained function can be determined by means of XGBoosting (Extreme Gradient Boosting). In particular, a neural network can be a deep neural network, a convolutional neural network or a convolutional deep neural network. Furthermore, a neural network can be an adversarial network, a deep adversarial network and/or a generative adversarial network. In particular, a neural network can be a recurrent neural network. In particular, a recurrent neural network can be a network with long short-term memory (LSTM), in particular, a gated recurrent unit (GRU). In particular, a trained function can comprise a combination of the described approaches. In particular, the approaches described here are specified for a trained function network architecture of the trained function.

In other embodiments, the first change information item can be transferred with the transmitting apparatus interface to the receiving apparatus interface.

In the method step of determining a first modified base image, the first modified base image is determined dependent upon the first change information item and the first base image. Therein the first base image is modified on the basis of the first change information item such that the modified first base image maps the changes according to the first change information item. In particular, the modified first base image corresponds to the second medical image. In particular, in the correct case, the modified first base image maps the same situation as the second medical image.

The first modified base image can therein be determined, in particular, by applying a trained function. The trained function can be configured as described above, in general, for a trained function. The trained function can therein be different from the above-described trained function for determining the first change information item. The trained function can therein be applied to the first base image and the first change information item. Therein, the first modified base image can be generated. In other words, the method step of determining the first modified base image can comprise a method step of using a trained function on the first base image and the first change information item.

In the method step of checking the second medical image and/or the first modified base image, the second medical image and/or the first modified base image is checked. In particular, it is checked whether a correct transfer of the second medical image can be ensured.

On checking the second medical image, it can be checked in particular whether the second medical image has or comprises an anomaly. For example, it can be checked whether a hemorrhage is discernible in the second medical image. In particular, it can be checked whether the second medical image deviates from a standard.

When the first modified base image is checked, it can be checked whether the first modified base image corresponds to the second medical image. In particular, it can therein be checked whether the first modified base image comprises all the medically relevant details of the second medical image.

A medically relevant detail can be, for example, a hemorrhage, a change in a perspective, an anatomical abnormality, an anomaly, etc.

In the method step of transferring the second medical image, the second medical image is transferred dependent upon the result of the method step of checking the second medical image and/or the first modified base image. Therein, the second medical image is transferred with the transmitting apparatus interface to the receiving apparatus interface. In particular, the second medical image is transferred if, in the method step of checking, an anomaly has been recognized or detected or determined in the second medical image. Alternatively or additionally, the second medical image is transferred if the first modified base image does not correspond to the second medical image. In other words, the second medical image can be transferred if the first modified base image does not comprise all the medically relevant details from the second medical image.

The inventor has discovered that, in this way, it can be ensured that in the event that a medical image cannot be transferred purely on the basis of change information, the medical image is transferred as such. In this way, it can be ensured that all the medically relevant details are transferred in a medical image. The transfer of the change information reduces the transferred data quantity in the event that in this way, all the medically relevant details can be transferred. The inventor has discovered that delays due to the transfer of the medical images from the transmitting apparatus to the receiving apparatus can thus be minimized.

According to one example embodiment, the method step of checking the second medical image and/or the first modified base image comprises a method step of determining a first similarity value between the first modified base image and the second medical image, and a method step of providing the first similarity value.

The first similarity value describes a similarity between the second medical image and the first modified base image. The first similarity value can comprise, in particular, a real number. The real number can describe and/or specify a similarity between the first modified base image and the second medical image. In particular, a deviation between the first modified base image and the second medical image can be derived from the similarity. Alternatively or additionally, the first similarity value can comprise a percentage value which specifies the similarity between the first modified base image and the second medical image. Alternatively or additionally, the first similarity value can specify a classification for the similarity between the first modified base image and the second medical image. For example, on the basis of the classification, the similarity can be divided into two classes, “similar” or “not similar” or “1” and “0”. Alternatively, the first similarity value can subdivide the similarity into more finely graduated classes.

If the second medical image and the first modified base image comprise voxel or pixel values, the first similarity value can depend, for example, on a sum of the squared deviations of the voxel or pixel values. Herein, it applies that the greater the sum of the squared deviations, the greater is the deviation between the first modified base image and the second medical image and/or the smaller is the similarity between the first modified base image and the second medical image.

Many alternative possibilities for determining the first similarity value are conceivable.

Alternatively, the first similarity value can comprise an information item regarding whether a medically relevant detail of the second medical image is included by the first modified base image and/or is mapped therein. In other words, the first similarity value can specify whether all the medically relevant details of the second medical image are mapped in the first modified base image. Alternatively or additionally, the first similarity value can specify how strongly the first modified base image deviates from the second medical image.

In the method step of determining the first similarity value, the first similarity value can be determined by applying a function to the second medical image and the first modified base image and/or the voxel and/or pixel values of the images.

In the method step of providing the first similarity value, the first similarity value is provided. In particular, the first similarity value is provided such that the method step of transferring the second medical image can depend upon the first similarity value. In other words, the first similarity value is provided such that a decision can be made, on the basis of and/or dependent upon the first similarity value, as to whether the second medical image is transferred or not.

The inventor has discovered that by way of the first similarity value, it can be determined whether the first modified base image meets all the safety standards. In other words, on the basis of the first similarity value, it can be checked whether it is sufficient to provide the first modified base image to the medical practitioner or whether the second medical image should be provided, since the first modified base image does not map all the medically relevant details and/or deviates too severely from the second medical image.

According to another example embodiment, in the method step of the first similarity value, the first similarity value is determined by applying a first trained function to the first modified base image and the second medical image.

The first trained function can be configured, in particular, as described in general above for a trained function.

The first trained function can be trained, in particular, by applying the first trained function to a plurality of pairs of first modified base images and second medical images. The result of the first trained function can therein be compared for every pair of a first modified base image and a second medical image with a predefined first similarity value. The trained function can be adapted during the training such that the result matches as well as possible with the predefined first similarity value. The result then corresponds to the first similarity value. The predefined first similarity value can have been determined, for example, manually. In particular, for every pair comprising a first modified base image and a second medical image, a predefined first similarity value for training the first trained function can be preset. The predefined first similarity value can be preset, for example, by the medical practitioner.

The inventor has discovered that with the first trained function, differences between the first modified base image and the second medical image, which do not lead to a deviation between the voxel and/or pixel values, can also be recognized. In particular, it is possible in this way to recognize lacking medically relevant details and/or anomalies in the first modified base image. In other words, it can be determined by means of the first trained function whether the first modified base image comprises all the medically relevant details and/or anomalies of the second medical image.

According to another example embodiment, the method steps of determining the first modified base image and determining and providing the first similarity value is carried out with the transmitting apparatus computing unit. The method therein comprises the following method steps if the first similarity value is greater than or equal to a first threshold value: a method step of transferring the first change information item with the transmitting apparatus interface to the receiving apparatus interface, a method step of determining the first modified base image with the receiving apparatus computing unit and a method step of providing the first modified base image with the receiving apparatus interface. The method therein comprises the following method steps if the first similarity value falls below the first threshold value: a method step of determining a second base image dependent upon the second medical image with the transmitting apparatus computing unit, a method step of transferring the second base image with the transmitting apparatus interface to the receiving apparatus interface and a method step of providing the second base image with the receiving apparatus interface.

In an example embodiment, the modified first base image is therefore determined with the transmitting apparatus. The method step of checking the second medical image and/or the first modified base image comprising the method steps of determining the first similarity value and providing the first similarity value are also carried out with the transmitting apparatus. Therein, “determined and/or carried out with the transmitting apparatus” means that the corresponding method step is carried out with the transmitting computing unit or the transmitting apparatus interface. In particular, the method steps of determining the first modified base image and determining the first similarity value are carried out with the transmitting apparatus computing unit. In particular, the method step of providing the first similarity value is carried out with the transmitting apparatus interface unit and/or with a sub-transmitting apparatus interface.

The first similarity value is compared with a first threshold value. The first threshold value is, in particular, predefined. The first threshold value specifies, in particular, a minimum similarity between the first modified base image and the second medical image that must be provided so that the medical safety standards are maintained.

The first threshold value can be, in particular, a real number. The threshold value is, in particular, a real number if the first similarity value is a real number.

Alternatively, the first threshold value can specify at least one class if the first similarity value comprises a classification. For example, with the first similarity value, the similarity between the first modified base image and the second medical image can be divided into two classes. According to the above example, the classes can be referred to, for example, as “1”, “0” or “similar” or “not similar”. The first threshold value can be, for example, “1” or “similar”. The first threshold value then divides the classes into two groups “1” and/or “similar” and “0” and/or “not similar”.

If the first similarity value is greater than or equal to the first threshold value, it can be assumed that the similarity between the first modified base image and the second medical image is sufficient to meet the safety standard. If the first similarity value is smaller than the first threshold value and/or falls below it, the first modified base image and the second medical image do not meet the requirements for the similarity. In particular, it cannot be assumed that all the medically relevant details from the second medical image are displayed and/or mapped in the first modified base image.

If the first similarity value is greater than or equal to the first threshold value, in the method step of transferring the first change information item, the first change information item is transferred with the transmitting apparatus interface to the receiving apparatus interface. Subsequently, in the method step of determining the first modified base image, the first modified base image is determined with the receiving apparatus computing unit. Therein, the first modified base image is determined as described above. In particular, the first modified base image is determined with the receiving apparatus computing unit similarly to the transmitting apparatus computing unit. The first modified base image is then provided with the receiving apparatus interface in the method step of providing the first modified base image. In particular, the first modified base image can be provided to the medical practitioner by means of the display unit of the receiving apparatus.

If the first similarity value falls below the first threshold value, in the method step of determining a second base image, the second base image is determined dependent upon the second medical image with the transmitting apparatus computing unit. The second base image is determined dependent upon the second medical image, like the first base image dependent upon the first medical image. The second base image can therein be, in particular, identical to the second medical image. Alternatively, the second base image can be a processed version of the second medical image. The processed version can be determined, in particular, through image processing and/or compression of the second medical image.

The second base image can replace the first base image in further method steps. In particular, the change information of the medical images of the plurality of medical images following temporally from the second medical image are then determined dependent upon the second base image. This applies until a new base image is determined again.

In the method step of transferring the second base image, the second base image is transferred with the transmitting apparatus interface to the receiving apparatus interface. Therein, the method step of transferring the second medical image dependent upon the result of the checking comprises the method step of transferring the second base image. Therein the result of the checking is the first similarity value. The second medical image is transferred in the method step in the form of the second base image.

In the method step of providing the second base image, the second base image is provided with the receiving apparatus interface. In particular, the second base image can be provided to the medical practitioner with the display unit of the receiving apparatus.

The inventor has discovered that before the transfer of the second medical image or the first change information item, it can be checked whether the first modified base image resembles the second medical image sufficiently to meet the safety standards. On the basis of this checking, the data can then be transferred that ensure that all the medically relevant details are provided to the medical practitioner. In the event that the first modified base image maps all the medically relevant details of the second medical image, it is sufficient to transfer the first change information item and in this way to reduce the data transfer. If this cannot be ensured, the second base image is transferred. The inventor has discovered that by way of the different embodiments of the method, dependent upon the first similarity value, it can be ensured that an image is always provided to the medical practitioner which comprises and/or maps all the medically relevant details of the second medical image. Therein, either the modified first base image or the second base image can be provided.

According to another example embodiment, the method comprises a method step of transferring the first change information item with the transmitting apparatus interface to the receiving apparatus interface. Therein, the method step of determining the first modified base image is carried out with the receiving apparatus interface. The method further comprises a method step of transferring the first modified base image with the receiving apparatus interface to the transmitting apparatus interface. Therein, the method steps of determining and providing the first similarity value are carried out with the transmitting apparatus. The method therein comprises the following method step if the first similarity value is greater than or equal to a first threshold value: a provision of the first modified base image with the receiving apparatus interface. The method further comprises the following method steps if the first similarity value falls below the first threshold value: a determination of a second base image dependent upon the second medical image with the transmitting apparatus computing unit, a transfer of the second base image with the transmitting apparatus interface to the receiving apparatus interface and a provision of the second base image with the receiving apparatus interface.

In an example embodiment, the first change information item is transferred from the transmitting apparatus to the receiving apparatus. In particular, the first change information item is transferred via the corresponding interfaces and/or sub-interfaces of the transmitting apparatus and the receiving apparatus.

The first modified base image is determined dependent upon the first change information item and the first base image by means of the receiving apparatus computing unit. In other words, the first modified base image is determined by the receiving apparatus.

The first modified base image is transferred in the method step of transferring of the first modified base image with the receiving apparatus interface to the transmitting apparatus interface. In other words, the first modified base image is transferred from the receiving apparatus to the transmitting apparatus.

The method steps of determining and providing the first similarity value are carried out with the transmitting apparatus and/or with the transmitting apparatus computing unit and the transmitting apparatus interface.

The first threshold value is configured, in particular, as described above. The first threshold value is compared with the first similarity value.

If the first similarity value is greater than or equal to the first threshold value, the first modified base image is provided with the receiving apparatus interface. In particular, the first modified base image is provided to the medical practitioner by means of the display unit of the receiving apparatus.

If the first similarity value falls below the first threshold value, the method step of determining a second base image is carried out dependent upon the second medical image with the transmitting apparatus computing unit. The second base image is configured, in particular, as described above. The second base image is determined dependent upon the second medical image similarly to the description of the determination of the first base image dependent upon the first medical image.

In the method step of transferring the second base image, the second base image is transferred from the transmitting apparatus interface to the receiving apparatus interface if the first similarity value falls below the first threshold value. In other words, the first base image is transferred from the transmitting apparatus to the receiving apparatus. The method step of transferring the second base image is therein included by the method step of transferring the second medical image dependent upon the result of the checking. The second medical image is thus transferred in the form of the second base image.

In the method step of providing the second base image, the second base image is provided with the receiving apparatus interface and/or a sub-receiving apparatus interface if the first similarity value falls below the first threshold value. In particular, the second base image is provided to the medical practitioner with the display unit of the receiving apparatus. In other words, the second base image is displayed to the medical practitioner.

As described above, the second base image replaces the first base image in subsequent passes of the method. In other words, a change information item of images following temporally from the second medical image is then determined dependent upon the second medical image and/or the second base image. According to the description for the first and second medical images, the second base image can be reduced, in turn, by a third base image if one of the above conditions is met.

The inventor has discovered that the first change information item can be transferred in the standard manner in order to be able to provide the medical practitioner at least always with the first modified base image. Thus, for example, on an interruption of the transfer, at least the first modified base image can be provided to the medical practitioner. The checking of the first modified base image and the method steps following therefrom can then be carried out with the transmitting apparatus. In this way, computing capacity can be reduced and/or saved on the receiving apparatus. The inventor has discovered that by way of the different embodiments of the method, dependent upon the first similarity value, it can be ensured that an image is always provided to the medical practitioner which comprises and/or maps all the medically relevant details of the first base image. Therein, either the modified first base image or the second base image can be provided.

According to another example embodiment, the method comprises a method step of transferring the first change information item with the transmitting apparatus interface to the receiving apparatus interface. Therein, the method step of determining the first modified base image is carried out with the receiving apparatus computing unit. The method further comprises a method step of transferring the second medical image with the transmitting apparatus interface to the receiving apparatus interface. Therein, the method steps of determining and providing the first similarity value are carried out with the receiving apparatus. The method comprises the following method step if the first similarity value is greater than or equal to a first threshold value: providing the first modified base image with the receiving apparatus interface. The method comprises the following method steps if the first similarity value falls below the first threshold value: determining a second base image dependent upon the second medical image with the receiving apparatus computing unit and providing the second base image with the receiving apparatus interface.

In the method step of transferring the first change information item, in an embodiment, the first change information item is transferred from the transmitting apparatus interface to the receiving apparatus interface. In other words, the first change information item is transferred from the transmitting apparatus to the receiving apparatus.

The method step of determining the first modified base image is therein carried out with the receiving apparatus computing unit. In other words, the first modified base image is carried out dependent upon the first base image and the first change information item with the receiving apparatus.

In the method step of transferring the second medical image, the second medical image is transferred with the transmitting apparatus interface to the receiving apparatus interface. In other words, the second medical image is transferred from the transmitting apparatus to the receiving apparatus. The method step of transferring the second base image is therein included by the method step of transferring the second medical image dependent upon the result of the checking.

The method steps of determining and providing the first similarity value are carried out in an embodiment with the receiving apparatus and/or with the receiving apparatus computing unit and the receiving apparatus interface.

The first threshold value is configured, in particular, as described above. In particular, the first threshold value is compared with the first similarity value.

The method comprises the following method step if the first similarity value is greater than or equal to the first threshold value: providing the first modified base image with the receiving apparatus interface. In particular, the first modified base image can be provided and/or displayed to the medical practitioner with the display unit of the receiving apparatus.

If the first similarity value falls below the first threshold value and/or is smaller than the first threshold value, the method comprises the method step of determining a second base image dependent upon the second medical image with the receiving apparatus interface. The method step of determining the second base image can be configured, in particular, as described above. The second base image can be configured, in particular, as described above. The method then additionally comprises the method step of providing the second base image with the receiving apparatus interface. In particular, the second base image is provided to the medical practitioner with the display unit of the receiving apparatus.

The inventor has discovered that the checking of the modified base image and the method steps following therefrom can be carried out or executed with the receiving apparatus and/or the receiving apparatus interface and the receiving apparatus computing unit. In particular, the inventor has discovered that the second medical base image can be determined directly with the receiving apparatus computing unit if the second medical image is transferred for checking from the transmitting apparatus to the receiving apparatus. The inventor has discovered that by way of the different embodiments of the method, dependent upon the first similarity value, it can be ensured that an image is always provided to the medical practitioner which comprises and/or maps all the medically relevant details of the first base image. Therein, either the modified first base image or the second base image can be provided.

According to another example embodiment, the plurality of medical images comprises a plurality of second medical images. Therein, the plurality of second medical images have been captured temporally after the first medical image. Therein, the second medical images are arranged in a temporal sequence. Therein, the method step of transferring the second medical image with the transmitting apparatus interface to the receiving apparatus interface and/or the method step of transferring the first modified base image from the receiving apparatus interface to the transmitting apparatus interface and the method steps dependent upon the first threshold value are carried out at predetermined intervals, in particular, for every tenth or every twentieth or every fiftieth second medical image. Therein, the method for all the other second medical images comprises a method step of providing the first modified base image with the receiving apparatus interface.

In particular, in at least some example embodiments, the method step of transferring the second medical image from the transmitting apparatus interface to the receiving apparatus interface and all the method steps dependent upon the first threshold value are carried out at predetermined intervals.

Alternatively, in at least one example embodiment, the method step of transferring the first modified base image from the transmitting apparatus interface to the receiving apparatus interface and all the method steps dependent upon the first threshold value are carried out at predetermined intervals.

The method steps dependent upon the first threshold value are therein, in particular, the method steps that are carried out according to the above description if the first similarity value is greater than or equal to the first threshold value or if the first similarity value falls below the first threshold.

The predetermined intervals specify for which nth second medical image the stipulated method steps are carried out. In particular, the specified method steps are carried out for every tenth or twentieth or fiftieth second medical image. In other words, the specified method steps are carried out for every tenth or twentieth or fiftieth second medical image.

Therein, all the method steps are carried out sequentially for the second medical images according to the temporal sequence of the second medical images.

For every second medical image for which the specified method steps are not carried out, the method step of providing the first modified base image is carried out with the receiving apparatus interface. In other words, every second medical image for which the specified method steps are not carried out is provided in the form of the first modified base image with the receiving apparatus interface. In particular, the first modified base image is therein provided and/or displayed to the medical practitioner, in particular, by means of the display unit of the receiving apparatus.

Thus, only in the predetermined intervals is it checked whether the first modified base image comprises all the medically relevant details of the second medical image. For all the other second medical images, the first modified base image is provided in the standard manner.

The inventor has discovered that the data quantity transferred can be reduced if checking is carried out only in the predetermined intervals whether the first modified base image comprises and/or maps all the medically relevant details of the second medical image. The inventor has discovered that this is sufficient to meet the medical safety standards.

According to another example embodiment, the method step of checking the second medical image and/or the first modified base image comprises a method step of applying a second trained function to the first and/or second medical image. Therein, it is determined whether the first and/or the second medical image has an anomaly. Therein, the method step of transferring the second medical image from the transmitting apparatus interface to the receiving apparatus interface is carried out if the first and/or the second medical image has an anomaly.

The second trained function can be configured, in particular, as described in general above for a trained function. The second trained function can be configured, in particular, to recognize an anomaly in a medical image. For this purpose, the second trained function can be applied to the corresponding medical image, in particular, to the first and/or second medical image. Therein, a result is generated which states whether the corresponding medical image has an anomaly. The designation “the corresponding medical image” stands in for “the first and/or second medical image”.

An anomaly can be, in particular, a medically relevant detail in the corresponding medical image. In particular, the anomaly describes a medically relevant detail in the corresponding image which is normally not found in a medical image. Alternatively or additionally, the anomaly describes a medically relevant detail in the corresponding medical image which indicates an anatomical abnormality of the patient and/or an abnormality in the medical intervention. For example, the anomaly can be a hemorrhage and/or a tumor and/or a blood clot, etc.

Alternatively, an anomaly can occur due to a changed perspective onto the portion of the patient in the corresponding medical image. For example, a C-arm can be moved during the capture of the plurality of medical images. Due to the movement, the perspective in the medical images onto the mapped portion of the patient is changed. The anomaly reveals that the images no longer fit one another. In other words, the anomaly can disclose that a difference between the second medical image and the first base image is too great.

The method step of transferring the second medical image from the transmitting apparatus interface to the receiving apparatus interface is carried out dependent upon the result of the checking if the first and/or the second medical image has an anomaly. Therein, the method step of transferring the second medical image is carried out if the second medical image has an anomaly.

The inventor has discovered that an anomaly possibly cannot be correctly transferred purely by way of the first change information item. The inventor has discovered that in this case, the second medical image and/or the second base image should be transferred directly in order to ensure that all the medically relevant details, in particular the anomaly, are included and/or mapped in the image provided.

According to another example embodiment, the second trained function has been trained with a predefined plurality of medical images and/or the second trained function comprises a long-short-term memory network.

The predefined plurality of medical images can, in particular, comprise at least one typical progression of the medical intervention. Alternatively or additionally, the predefined plurality of medical images can typically comprise and/or map images of the portion of the patient from a plurality of patients, said portion being mapped in the first and/or second medical image.

In embodiments, the predefined plurality of medical images also comprise at least one medical image that has an anomaly. In particular, the medical images of the predefined plurality of medical images can be annotated. In other words, the medical images of the plurality of medical images which comprise and/or have an anomaly can then be identified.

For training the second trained function, the second trained function is applied to the predefined plurality of medical images. It is therein determined whether the medical images of the predefined plurality of medical images have an anomaly. In particular, the individual medical image for each of the predefined plurality of medical images is determined.

If the medical images of the predefined plurality of medical images is not annotated, the medical images can be clustered by applying the second trained function. A medical image that has an anomaly can then be assigned to no cluster or to a cluster to which medical images with anomalies are assigned. In particular, the second trained function can be trained by means of unsupervised learning.

If the medical images of the predefined plurality of medical images are annotated, the result of the second trained function is compared, after the application to the predefined plurality of medical images, with the annotation. The first trained function is adapted such that the result matches as well as possible with the annotation. In particular, in this way, the second trained function can be trained by means of supervised learning.

Alternatively or additionally, the second trained function can comprise a long-short-term memory (LSTM) network. On the basis of such a network, temporal sequences can be analyzed. An LSTM network can analyze, for example, a temporal sequence of medical images. For this purpose, the second trained function can be trained on a typical sequence of medical images. For this purpose, the predefined plurality of medical images can comprise at least one typical sequence of the medical intervention in the form of a temporal sequence of medical images. In particular, by applying the second trained function to the first and/or second medical image, it can be detected whether the first and/or second medical image corresponds to a medical image in the typical sequence. In other words, the temporal sequence of the plurality of medical images can be monitored and, given an abnormality, it can be output that the corresponding medical image has an anomaly.

The inventor has discovered that the second trained function can be trained on a predefined plurality of medical images. The inventor has discovered that, for this purpose, no, or only a small, manual effort is necessary for annotating the medical images of the predefined plurality of medical images. The inventor has discovered that the second trained function can be based upon different networks. The inventor has discovered that the advantages of the different networks in the second trained function can be combined. The cluster analysis offers the advantage that training can take place on temporally unconnected medical images without annotation. The monitored learning offers the advantage that the training of the second trained function can be monitored. The LSTM network offers the advantage that deviations from a typical temporal progression and/or a typical temporal sequence can be recognized.

According to another example embodiment, the method comprises a method step of determining a second base image dependent upon the second medical image with the transmitting apparatus computing unit if the first and/or second medical image has an anomaly. The method then further comprises a method step of transferring the second base image with the transmitting apparatus interface to the receiving apparatus interface. The method then also comprises a method step of providing the second base image with the receiving apparatus interface.

In the method step of determining the second base image, the second base image is determined as described above. Therein, the second base image is configured as described above.

In the method step of transferring the second base image, the second base image is transferred with the transmitting apparatus interface to the receiving apparatus interface. In other words, the second base image is transferred by the transmitting apparatus to the receiving apparatus. The method step of transferring the second base image is therein included by the method step of transferring the second medical image dependent upon the result of the checking. In particular, the second medical image is transferred in the form of the second base image.

In the method step of providing the second base image, the second base image is provided with the receiving apparatus interface. In particular, the second base image can be provided to the medical practitioner by means of the display unit of the receiving apparatus.

The second medical base image can then replace the first medical base image as described above.

The inventor has discovered that if the first medical image and/or the second medical image has an anomaly, the second base image should be provided in order to ensure that all the medically relevant details, in particular the anomaly, are included and/or mapped by the image provided. The inventor has discovered that under some circumstances, the first change information item is then not sufficient to map the anomaly and the second medical image in the form of the second medical base image should be transferred completely.

According to another example embodiment, the method comprises a method step of determining a second similarity value between the first base image and the second medical image with the transmitting apparatus computing unit. The method also comprises the following method steps if the second similarity value falls below a second threshold value: determining a second base image dependent upon the second medical image with the receiving apparatus computing unit, transferring the second base image by means of the transmitting apparatus interface to the receiving apparatus interface and providing the second base image with the receiving apparatus interface. The method also comprises the following method steps if the second similarity value is greater than or equal to the second threshold value: transferring the first change information item by means of the transmitting apparatus interface to the receiving apparatus interface, wherein the modified first base image is determined with the receiving apparatus interface, and providing the modified first base image with the receiving apparatus interface.

The second similarity value describes a similarity between the first base image and the second medical image. The second similarity value can be determined similarly to the first similarity value. For example, the second similarity value can depend upon a sum of the squared deviations between the voxel or pixel values of the first base image and the second medical image if the first base image and the second medical image comprise voxel or pixel values. Alternatively, the second similarity value can describe a structural similarity between the first base image and the second medical image. For example, the second similarity value can comprise a result of a comparison of the contrast of the first base image and the second medical image. Alternatively, the second similarity value can comprise a result of a comparison of a histogram of the voxel or pixel values of the first base image and the second medical image.

The second similarity value can comprise, for example, a real number, a percentage value and/or a classification as described above for the first similarity value.

In the method step of determining the second similarity value, the second similarity value is determined by means of the transmitting apparatus computing unit. In other words, the second similarity value is determined with the transmitting apparatus.

The second similarity value is compared with the second threshold value. The second threshold value specifies which value the second similarity value must assume at least, so that the first base image can apply as the basis for the second medical image. In other words, the second threshold value specifies which value the second similarity value must assume at least, so that it can be assumed that the first modified base image, determined dependent upon the first base image and the first change information item, comprises and/or maps all the medically relevant details of the second medical image.

If the second similarity value falls below the second threshold value, the method steps of determining the second base image, of transferring the second base image and providing the second base image are carried out as described above for the other embodiments. In other words, the specified method steps are carried out if the second similarity value is smaller than the second threshold value. The method step of determining the second base image is therein carried out by the transmitting apparatus computing unit. In the method step of transferring the second base image, the second base image is transferred by means of the transmitting apparatus interface to the receiving apparatus interface. The provision of the second base image takes place by means of the receiving apparatus interface. The second base image is configured, in particular, as described above.

If the second similarity value is greater than or equal to the second threshold value, the method steps of transferring the first change information item and providing the modified first base image are carried out as described above for the other embodiments. In the transfer of the first change information item, the first change information item is transferred by means of the transmitting apparatus interface to the receiving apparatus interface. The modified first base image is then determined by means of the receiving apparatus computing unit. The modified first base image is provided by means of the receiving apparatus interface. The first change information item and the modified first base image are therein configured as described above.

With a combination of the embodiments described above which comprise the determination of the first and second similarity value, the case is preferably treated that arises if one of the similarity values falls below the corresponding threshold value. In other words, if the first similarity value falls below the first threshold value, the associated method steps are carried out regardless of how the second similarity value behaves in relation to the second threshold value. Similarly, if the second similarity value falls below the second threshold value, the associated method steps are carried out regardless of how the first similarity value behaves in relation to the first threshold value. If both similarity values fall below the associated threshold value, the method steps then carried out are the same for all cases.

The inventor has discovered that the first modified base image only corresponds to the second medical image and/or maps all the medically relevant details of the second medical image if the first base image and the second medical image are similar to each other at least to a particular degree. This degree can be specified by means of the second threshold value. The similarity between the first base image and the second medical image is therein mapped by the second similarity value. The inventor has discovered that in the event that the first base image and the second medical image are not sufficiently similar, the first modified base image possibly does not comprise or map all the medically relevant details of the second medical image. The inventor has discovered that the second medical image should then be transferred completely in the form of the second base image. The inventor has discovered that the second base image then replaces the first base image.

According to another example embodiment, in the method step of determining the second similarity value, the second similarity value is determined by applying a third trained function to the first base image and the second medical image.

The third trained function can therein be configured as described above in general for a trained function.

For training the third trained function, the third trained function can be applied to a predefined plurality of first base images and second medical images. Therein, the third trained function can be based, for example, on a k-means clustering and/or alternative cluster analyses. Alternatively or additionally, a second similarity value can be determined manually in each case for a pair comprising a first base image and a second medical image of the predefined plurality of first base images and second medical images. For training the third trained function, the third trained function can be adapted in such a way that the result determined by applying the third trained function to the predefined plurality of first base images and the plurality of second medical images corresponds as well as possible to the manually determined second similarity values and/or matches them as well as possible. This can be designated “supervised learning”.

The inventor has discovered that the second similarity value can be determined by means of the third trained function. The inventor has discovered that thereby relative deviations of the voxel or pixel values which would possibly influence the sum of the squared intervals of the voxel or pixel values can be ignored. The inventor has discovered that such deviations can have a large influence on the sum of the squared intervals.

According to another example embodiment, the plurality of medical images comprises at least one third medical image. Therein, the third medical image has been captured temporally after the second medical image. The method therein comprises a method step of determining a second change information item dependent upon the first and the third medical image with the transmitting apparatus computing unit. The method therein additionally comprises a method step of determining a third similarity value between the first and the second change information item with the transmitting apparatus computing unit. The method therein comprises a method step of providing the third similarity value with the transmitting apparatus interface if the third similarity value falls below a third threshold value.

The third medical image is configured similarly to the first and the second medical image.

In the method step of determining the second change information item, the second change information item is determined according to the description relating to the determination of the first change information item. The second change information item therein describes, in particular, a change between the first medical image and the third medical image. In particular, on the basis of the first base image and the third medical image, a modified second base image can be determined. Therein, the modified second base image advantageously comprises all the medically relevant details of the third medical image.

If the first base image has been replaced in one of the preceding method steps by the second medical base image, the second change information item is determined dependent upon the second medical image and the third medical image. The first base image is then replaced, in particular, by the second base image if the second base image is determined and transferred.

In the method step of determining the third similarity value, the third similarity value is determined by means of the transmitting apparatus computing unit. Therein, the third similarity value describes a similarity between the first and the second change information items. In other words, it can be derived from the third similarity value how strongly the first and second change information item deviate from one another.

The third similarity value can comprise, for example, the similarity in the form of a real number, a percentage value and/or a classification as described above for the first similarity value.

In the method step of providing the third similarity value, the third similarity value is determined by means of the transmitting apparatus interface. In particular, the method step of providing the third similarity value can comprise a method step of transferring the third similarity value with the transmitting apparatus interface to the receiving apparatus interface. The receiving apparatus interface can then provide the third similarity value. In particular, the receiving apparatus interface can provide the third similarity value to the medical practitioner. In particular, the third similarity value can be provided to the medical practitioner in the form of a set of traffic lights. In particular, the third similarity value can be displayed to the medical practitioner by means of the display unit of the receiving apparatus. The third similarity value is then provided, in particular, if it falls below the third threshold value. In other words, the third similarity value is provided if it is lower than the third threshold value. In particular, for example, the traffic lights can then show red. In other words, the medical practitioner is informed if the third similarity value falls below the third threshold value. The third threshold value specifies from when the third similarity value should be provided. The third threshold value specifies when a deviation between the first change information item and the second change information item is so great that the third similarity value should be provided.

The inventor has discovered that the medical practitioner can recognize, on the basis of the third similarity value, when non-normal changes occur between two images. In other words, the medical practitioner can recognize, on the basis of the third similarity value, when two successive images are less similar to each other than is usual. This can be caused, for example, by a suddenly occurring hemorrhage and/or an unwanted movement of a medical technology device, in one of the images, etc. The inventor has discovered that the third similarity value should be provided in the event of an undershooting of the third threshold value, in order to clarify whether problems have occurred. This is necessary, in particular, if the medical practitioner is positioned spatially separated from the patient.

In an embodiment, the medical practitioner can demand that the third medical image is transferred, in particular, in the form of a third base image from the transmitting apparatus interface to the receiving apparatus interface if the third similarity value falls below the third threshold value. In this way, the medical practitioner can check whether an error has occurred during the determination of the second change information item or whether the third medical image actually deviates so severely from the second medical image.

According to another example embodiment, in the method step of determining the third similarity value, the third similarity value is determined by applying a fourth trained function to the first and second change information items.

The fourth trained function can therein be configured as described above in general for a trained function. In particular, the fourth trained function can be trained by applying the fourth trained function to a plurality of first change information items and a plurality of second change information items. In particular, the fourth trained function can be based upon a cluster analysis. Alternatively or additionally, the fourth trained function can be trained by means of “supervised learning”.

The inventor has discovered that the third similarity value can be determined by means of a fourth trained function. The inventor has discovered that the fourth trained function can also operate with abstract data such as the first and second change information items.

According to another example embodiment, the method further comprises a method step of determining a fourth similarity value between the first medical image and the second medical image with the transmitting apparatus computing unit. The method further comprises a method step of providing the fourth similarity value with the transmitting apparatus interface if the fourth similarity value falls below a fourth threshold value.

The fourth similarity value describes a similarity between the first medical image and the second medical image. The fourth similarity value is determined in the method step of determining the fourth similarity value with the transmitting apparatus computing unit. The fourth similarity value can therein be determined as described in relation to the first similarity value.

The fourth similarity value can comprise, for example, the similarity in the form of a real number, a percentage value and/or a classification as described above for the first similarity value.

In embodiments, the method step of determining the fourth similarity value can be included in the method step of applying the second trained function to the first and/or second medical image. In particular, the fourth similarity value can state whether the second medical image has an anomaly as compared with the first medical image. Alternatively, the fourth similarity value can state whether the first medical image has an anomaly as compared with the second medical image.

In the method step of providing the fourth similarity value, the fourth similarity value is determined with the transmitting apparatus interface, if the fourth similarity value falls below the fourth threshold value.

Therein, the fourth threshold value specifies when the similarity between the first and the second medical image is so low that the fourth similarity value should be provided.

The method step of providing the fourth similarity value can comprise, in particular, a method step of transferring the fourth similarity value from the transmitting apparatus interface to the receiving apparatus interface. In particular, the fourth similarity value can be provided to the medical practitioner with the receiving apparatus interface. In particular, the fourth similarity value can be provided to the medical practitioner by means of the display unit of the receiving apparatus. Alternatively or additionally, the fourth similarity value can be provided to the medical practitioner as described in relation to the third similarity value, by means of a set of traffic lights. In particular, the traffic lights can signal red or switch to “red” if the fourth similarity value falls below the fourth threshold value. With a combination of the embodiments, the traffic lights can light red at least if the third or fourth similarity value falls below the third and/or fourth threshold value.

The inventor has discovered that an excessively large deviation between the first and the second medical image can indicate a faulty capture of the medical images. The inventor has discovered that the medical practitioner should be notified thereof. An excessively large deviation is characterized in that the fourth similarity value falls below the fourth threshold value. The inventor has further discovered that an excessively large deviation between the first and the second medical image can lead thereto that not all the medically relevant details based upon the first base image and the first change information item are mapped in the modified first base image. The inventor has discovered that the medical practitioner can be notified thereof by the provision of the fourth similarity value.

According to another example embodiment, in the method step of determining the fourth similarity value, the fourth similarity value is determined by applying a fifth trained function to the first medical image and the second medical image.

The fifth trained function can therein be configured, in particular, as described above, in general, for a trained function. If the first medical image is identical to the first base image, the fifth trained function corresponds to the third trained function.

In the embodiments in which the method step of determining the fourth similarity value is included by the method step of applying the second trained function to the first and/or second medical image, the fifth trained function can correspond to the second trained function and/or include the second trained function.

The inventor has discovered that a similarity between the first and the second medical images can be determined in the form of the fourth similarity value by means of the fifth trained function. The inventor has discovered that the fifth trained function can also correctly determine the fourth similarity value if the voxel or pixel values of the first and second medical images deviate from one another, for example, due to different exposures.

At least one example embodiment further relates to a transmitting method comprising method steps according to one of the aspects and/or embodiments described above, which are carried out by the transmitting apparatus.

The transmitting method therein comprises the method steps that are carried out by a unit included by the transmitting apparatus. In particular, the transmitting method comprises those method steps which are carried out by the transmitting apparatus computing unit and by the transmitting apparatus interface and/or their sub-units.

At least one example embodiment also relates to a receiving method comprising method steps according to one of the aspects and/or embodiments described above, which are carried out by the receiving apparatus.

The receiving method therein comprises, in particular, the method steps that are carried out by a unit included by the receiving apparatus. In particular, the receiving method comprises those method steps which are carried out by the receiving apparatus computing unit and by the receiving apparatus interface and/or their sub-units.

At least one example embodiment further comprises a transmitting apparatus comprising a transmitting apparatus interface and a transmitting apparatus computing unit. Therein, the transmitting apparatus is configured to carry out a transmitting method.

In particular, the transmitting apparatus is configured to carry out the transmitting method described above comprising the method steps described above.

At least one example embodiment further relates to a receiving apparatus comprising a receiving apparatus interface and a receiving apparatus computing unit. Therein, the receiving apparatus is configured to carry out a receiving method.

In particular, the receiving apparatus is configured to carry out the receiving method described above comprising the method steps described above.

At least one example embodiment further relates to a transfer system comprising a transmitting apparatus and a receiving apparatus. Therein, the transmitting apparatus and the receiving apparatus are configured to carry out the method described above.

In particular, the transmitting apparatus is configured to carry out the transmitting method described above. In particular, the receiving apparatus is configured to carry out the receiving method described above.

A transfer system of this type can be configured, in particular, to carry out the method described above and its aspects for transferring a plurality of medical images from a transmitting apparatus comprising a transmitting apparatus interface and a transmitting apparatus computing unit to a receiving apparatus comprising a receiving apparatus interface and a receiving apparatus computing unit. The transfer system is configured to carry out this method and its aspects in that the transmitting apparatus interface, receiving apparatus interface, transmitting apparatus computing unit and receiving apparatus computing unit described above are configured as described above to carry out the corresponding method steps.

At least one example embodiment also relates to a computer program product having a computer program, and also a computer-readable medium. A realization largely through software has the advantage that conventionally used transmitting apparatuses and/or receiving apparatuses can also easily be upgraded by way of a software update in order to operate in the manner described. Such a computer program product can comprise, apart from the computer program, possibly additional constituents, such as, for example, documentation and/or additional components as well as hardware components, for example, hardware keys (dongles, etc.) for using the software.

At least one example embodiment relates also, in particular, to a computer program product with a computer program which can be loaded directly into a memory store of a transmitting system and/or a receiving apparatus, having program portions in order to carry out all the steps of the method described above for transferring a plurality of medical images, and its aspects, when the program portions are executed by the transmitting apparatus and/or the receiving apparatus.

At least one example embodiment relates also, in particular, to a computer-readable storage medium on which program portions readable and executable by a transmitting apparatus and/or a receiving apparatus are stored, in order to carry out all the steps of the method described above for transferring a plurality of medical images, and its aspects, when the program portions are executed by the transmitting apparatus and/or the receiving apparatus.

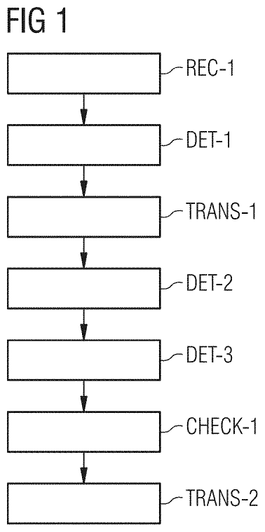

FIG. 1 shows a first exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

In the method step of receiving REC-1 a plurality of medical images, the plurality of medical images is received with the transmitting apparatus interface SSYS.IF.

The plurality of medical images comprises a temporal sequence of medical images. The plurality of medical images therein comprises at least one first medical image and one second medical image. Therein, the first medical image has been captured temporally before the second medical image. The plurality of medical images is captured with a medical technology device. The medical technology device can be, for example, an X-ray device, a computed tomography (CT) device, a C-arm, a magnetic resonance tomography (MRT) device, an ultrasound device, an angiography system, an electrocardiography system, a positron emission tomography (PET) system and/or a single photon emission computed tomography (SPECT) system. The plurality of the medical images therein maps at least a portion of a patient. Therein, the first medical image can map a different portion of the patient from the second medical image. In particular, the first and the second medical image map, at least partially, the same portion of the patient. Therein, the patient is a human. Alternatively, the patient can be an animal or an object. The plurality of medical images can be captured, in particular, during a medical intervention. The medical intervention is then image-assisted. Therein, the medical intervention is carried out by a medical practitioner on the basis of the plurality of medical images. The medical practitioner is therein positioned at the receiving apparatus RSYS. In particular, the medical practitioner carries out the medical intervention by means of the receiving apparatus RSYS. In other words, the medical practitioner carries out the medical intervention “remotely”.

The plurality of medical technology images can be received from the medical technology device by means of the transmitting apparatus interface SSYS.IF. Alternatively, the transmitting apparatus interface SSYS.IF can receive the plurality of medical images from a database. The database can be, for example, a picture archiving and communication system (PACS). Alternatively, the medical technology device can be the transmitting apparatus SSYS.

In the method step of determining DET 1 a first base image, the first base image is determined dependent upon the first medical image with the transmitting apparatus computing unit SSYS.CU. The first base image therein corresponds to the first medical image. Alternatively, the first base image can be a processed version of the first medical image. For example, the first medical image can be processed for determining DET-1 the first base image by means of image processing. The image processing can comprise, for example, a compression, a noise reduction, a contrast enhancement and/or an edge enhancement, etc. If the first base image comprises a processed version of the first medical image, the first medical image can be processed such that the first base image comprises a smaller data volume and/or a smaller data quantity than the first medical image.

In the method step of transferring TRANS-1 the first base image, the first base image is transferred with the transmitting apparatus interface SSYS.IF to the receiving apparatus interface RSYS.IF. The transmitting apparatus SSYS and the receiving apparatus RSYS can therein be arranged spatially separate from one another. For example, the transmitting apparatus SSYS and the receiving apparatus RSYS can be arranged spatially separated in the same room or in different rooms or in different buildings or in different cities or in different countries.

In the method step of determining DET-2 a first change information item, the first change information item is determined dependent upon the first and second medical images with the transmitting apparatus computing unit SSYS.CU. The first change information item describes what has changed between the first and the second medical images. The first change information item is therein configured such that, on the basis of the first medical image and the first change information item, the second medical image can be determined. The first change information item can comprise, in particular, at least a change point and/or a change vector which describes a change between the first medical image and the second medical image. The first change information item can be determined by means of a trained function. In particular, the first change information item can be determined by applying the trained function to the first and second medical images.

In at least one example embodiment, the first change information item can be transferred with the transmitting apparatus interface SSYS.IF to the receiving apparatus interface RSYS.IF.

In the method step of determining DET-3 a first modified base image, the first modified base image is determined dependent upon the first change information item and the first base image. The first modified base image can therein be determined by means of the transmitting apparatus computing unit SSYS.CU and/or the receiving apparatus computing unit RSYS.CU. The first modified base image therein advantageously comprises all the medically relevant details of the second medical image. In other words, all the medically relevant details of the second medical image are advantageously mapped in the first modified base image. A medically relevant detail can be, for example, a hemorrhage, a change in a perspective, an anatomical abnormality, an anomaly, etc. In particular, the first modified base image can correspond to the second medical image. The first modified base image can therein be determined, in particular, by applying a trained function to the first change information item and the first base image.

In the method step of checking CHECK-1 the second medical image and/or the first modified base image, the second medical image and/or the first modified base image is checked for medical correctness and/or medical abnormalities.

On checking CHECK-1 the second medical image, it can be checked in particular whether the second medical image comprises an anomaly and/or whether, for example, the second medical image corresponds to a standard. In other words, it can be checked whether the second medical image is typical for a standard progression of a medical intervention or whether an abnormality and/or anomaly is to be seen in the second medical image. In particular, the second medical image can be checked dependent upon the first medical image. For example, it can be checked whether unexpected changes occur between the first and second medical images.

During the checking CHECK-1 of the first modified base image, it is checked in particular, whether the first modified base image comprises all the medically relevant details of the second medical image. It can be checked, in particular, whether the first modified base image corresponds to the second medical image.

In the method step of transferring TRANS-2 the second medical image with the transmitting apparatus interface SSYS.IF to the receiving apparatus interface RSYS.IF, the second medical image is transferred dependent upon the result of the method step of checking CHECK-1 the second medical image and/or the first modified base image. In particular, the second medical image can then be transferred if on checking CHECK-1, it has been established that the second medical image comprises an anomaly and/or does not meet the standard. Alternatively or additionally, the second medical image can be transferred if the first modified base image does not comprise all the medically relevant details of the second medical image.

In particular, the second medical image is not transferred if the first modified base image comprises all the medically relevant details of the second medical image and/or if the first modified base image corresponds to the second medical image. In particular, in some embodiments, merely the first change information item can be transferred and the first modified base image can be determined with the receiving apparatus computing unit RSYS.CU.

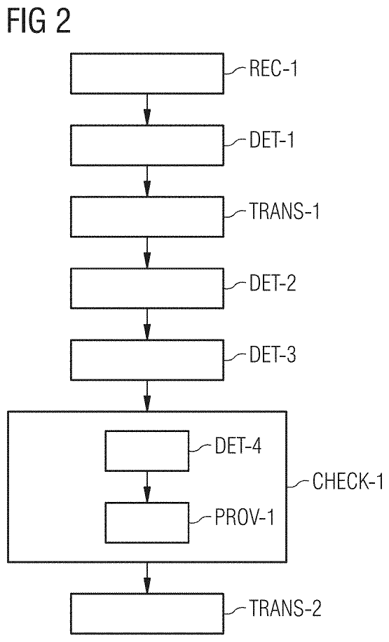

FIG. 2 shows a second exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1.

In at lest one example embodiment, the first exemplary embodiment as described in relation to the first exemplary embodiment can also comprise a method step of transferring the first change information item with the transmitting apparatus interface SSYS.IF to the receiving apparatus interface RSYS.IF. The first change information item can be transferred, in particular, if the second medical image is not transferred.

The method step of checking CHECK-1 comprises in this exemplary embodiment a method step of determining DET-4 a first similarity value SV_1 and a method step of providing PROV-1 the first similarity value SV_1.

In the method step of determining DET-4 the first similarity value SV_1, the first similarity value SV_1 is determined between the first modified base image and the second medical image. The first similarity value SV_1 thus describes a similarity between the first modified base image and the second medical image. The following apply herein: the larger the first similarity value SV_1, the more similar is the first modified base image to the second medical image. In particular, it can be derived from the first similarity value SV_1 how strongly the first modified base image deviates from the second medical image.

If the first modified base image and the second medical image comprise a plurality of voxel or pixel values, the first similarity value SW_1 can depend upon the sum of the squared intervals of the voxel or pixel values. The first similarity value SV_1 can then be calculated as follows:

$$S {V}_{1} = \frac{1}{{\sum }_{i = 1}^{N} {( {x}_{i} - {y}_{i} )}^{2}} .$$

Therein, the first modified base image and the second medical image comprise N pixels or voxels. Therein xᵢ are the voxel or pixel values of the first modified base image and yᵢ are the voxel or pixel values of the second medical image. Therein, SV₁ corresponds to the first similarity value SV_1.

In alternative embodiments, many further possibilities for determining the first similarity value on the basis of a distance or similarity value are conceivable.

Alternatively or additionally, the first similarity value SV_1 can be determined by applying a first trained function to the first modified base image and the second medical image. By applying the first trained function, the first similarity value SV_1 can be determined, for example, independently of the absolute voxel or pixel values. For example, the first similarity value SV_1 can specify a percentage match between the first modified base image and the second medical image. Alternatively or additionally, the first similarity value SV_1 can specify whether the first modified base image comprises all the medically relevant details of the second medical image. In particular, by means of the first similarity value SV_1, the similarity between the first modified base image and the second medical image can be classified. For example, a first similarity value SV_1 of “1” can specify that the first modified base image is similar to the second medical image and/or comprises all the medically relevant details. A first similarity value SV_1 of “0” can specify that the first modified base image is not similar to the second medical image and/or does not comprise all the medically relevant details.

In the method step of providing PROV-1 the first similarity value SV_1, the first similarity value SV_1 is provided for a further processing. In embodiments, the first similarity value SV_1 can be provided to the medical practitioner. Alternatively or additionally, further method steps can depend upon the first similarity value SV_1. Therein, the first similarity value SV_1 can be provided, in particular, by means of the transmitting apparatus interface SSYS.IF and/or the receiving apparatus interface RSYS.IF.

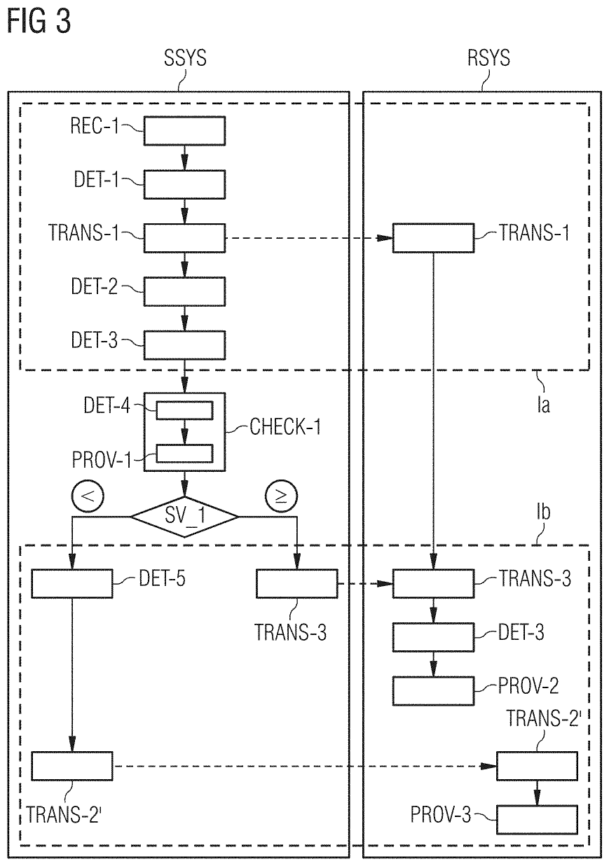

FIG. 3 shows a third exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1. The method steps of determining DET-4 the first similarity value SV_1 and providing PROV-1 the first similarity value SV_1 are configured according to the description relating to FIG. 2.

In the representation of the exemplary embodiment according to FIG. 3, all the method steps SSYS carried out by the transmitting apparatus are represented in the box on the left side of the figure. All the method steps carried out by the receiving apparatus RSYS are shown in the box on the right side of the figure. The method steps carried out by the transmitting apparatus SSYS are carried out, in particular, by means of the transmitting apparatus interface SSYS.IF and/or by means of the transmitting apparatus computing unit SSYS.CU. The method steps carried out by the receiving apparatus RSYS are carried out, in particular, by means of the receiving apparatus interface RSYS.IF and/or by means of the receiving apparatus computing unit RSYS.CU.

In the exemplary embodiment, the method steps of determining DET-3 the first modified base image and determining DET-4 the first similarity value SV_1 are carried out with the transmitting apparatus computing unit SSYS.CU. The method step of providing PROV-1 the first similarity value SV_1 is carried out with the transmitting apparatus interface SSYS.IF.

A first threshold value is predefined that determines at what first similarity value SV_1 it can be assumed that the first modified base image is sufficiently similar to the second medical image in order to be provided, in particular, to the medical practitioner, in particular, to carry out the medical intervention. If the first similarity value SV_1 falls below the first threshold value, it can be assumed that the deviation between the first modified base image and the second medical image is possibly so large that the first modified base image possibly does not comprise all the medically relevant details of the second medical image.

If the first similarity value SV_1 is larger than or equal to the first threshold value, the following method steps are carried out: transferring TRANS-3 the first change information item with the transmitting apparatus interface SSYS.IF to the receiving apparatus interface RSYS.IF, determining DET-3 the first modified base image with the receiving apparatus computing unit RSYS.CU and providing PROV-2 the first modified base image with the receiving apparatus interface RSYS.IF.

In the method step of transferring TRANS-3 the first change information item, the first change information item is transferred from the transmitting apparatus SSYS to the receiving apparatus RSYS, in particular by means of the corresponding interfaces.

Dependent upon the first base image and the first change information item, in the method step of determining DET-3 the first modified base image, the first modified base image is determined by means of the receiving apparatus computing unit RSYS.CU. In other words, the first modified base image is determined by the receiving apparatus RSYS.

In the method step of providing PROV-2 the first modified base image, the first modified base image is provided with the receiving apparatus interface RSYS.IF. The method step of providing PROV-2 the first modified base image can comprise, in particular, a display of the modified base image. For this purpose, the receiving apparatus interface RSYS.IF can comprise a display unit. By means of the display unit, the first modified base image is provided and/or displayed to the medical practitioner.

If the first similarity value SV_1 is larger than or equal to the first threshold value, the following method steps are carried out: determining DET-5 a second base image, transferring TRANS-2′ the second base image, providing PROV-3 the second base image.

In the method step of determining DET-5 the second base image, the second base image is determined with the transmitting apparatus computing unit SSYS.CU. The second base image is determined dependent upon the second medical image. The second base image is determined similarly to the first base image. The second base image can, in particular, correspond to the second medical image. Alternatively, the second base image can be a processed version of the second medical image.

In the method step of transferring TRANS-2′ the second base image, the second base image is transferred from the transmitting apparatus SSYS to the receiving apparatus RSYS. Therein, the second base image is transferred by means of the corresponding interfaces of the apparatuses. The method step of transferring TRANS-2′ the second base image is therein included by the method step of transferring TRANS-2 the second medical image. In other words, the second medical image is transferred in the form of the second base image. In other words, the method step of transferring TRANS-2′ the second base image corresponds to an execution of the method step of transferring TRANS-2 the second medical image.

On repeated execution of the method, the second base image replaces the first base image. A change information item of a further medical image is thus determined dependent upon the second and further medical image.

In the method step of providing PROV-3 the second base image, the second base image is provided by means of the receiving apparatus interface RSYS.IF. In particular, the provision PROV-3 of the second base image can take place similarly to the description of the method step of providing PROV-2 the first modified base image. In other words, the provision PROV-3 of the second base image can comprise a display of the second base image by means of the display unit. The second base image is provided to the medical practitioner in this way.

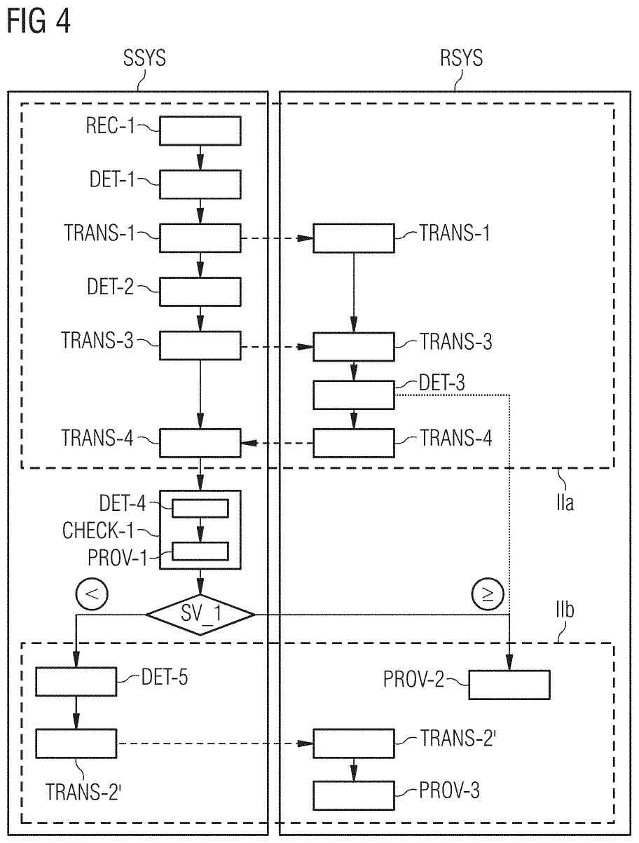

FIG. 4 shows a fourth exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1. The method steps of determining DET-4 the first similarity value SV_1 and providing PROV-1 the first similarity value SV_1 are configured according to the description relating to FIG. 2. The method steps of transferring TRANS-3 the first change information item, providing PROV-2 the first modified base image, determining DET-5 the second base image, transferring TRANS-2′ the second base image and providing PROV-3 the second base image are configured according to the description relating to FIG. 3.

In the exemplary embodiment, the first change information item is transferred independently of the first threshold value and the first similarity value SV_1 in the method step of transferring TRANS-3 the first change information item.

Dependent upon the first modified base image and the first change information item, in the method step of determining DET-3 the first modified base image, the first modified base image is determined with the receiving apparatus computing unit RSYS.CU.

In the method step of transferring TRANS-4 the first modified base image, the first modified base image is transferred from the receiving apparatus RSYS to the transmitting apparatus SSYS. Therein, the first modified base image is transferred by means of the transmitting apparatus interface SSYS.IF and the receiving apparatus interface RSYS.IF.

The method steps of determining DET-4 the first similarity value SV_1 and providing PROV-1 the first similarity value SV_1 are carried out by means of the transmitting apparatus SSYS. In particular the method step of determining DET-4 the first similarity value SV_1 with the transmitting apparatus computing unit SSYS.CU and the method step of providing PROV-1 the first similarity value SV_1 are carried out with the transmitting apparatus interface SSYS.IF.

If the first similarity value SV_1 is greater than or equal to the first threshold value, the first modified base image is provided by means of the receiving apparatus interface as described above in the method step of providing PROV-2 the first modified base image.

If the first similarity value SV_1 falls below the first threshold value, the method steps of determining DET-5 the second base image, transferring TRANS-2′ the second base image and providing PROV-3 the second base image are carried out as described above.

In embodiments, the plurality of medical images comprises a plurality of second medical images. The plurality of second medical images have therein been captured temporally after the first medical image. The method steps described above are therein carried out for each of the second medical images. In particular, the method steps of receiving REC-1 the plurality of medical images, determining DET-1 the first base image, transferring TRANS-1 the first base image, determining DET-2 the first change information item, transferring TRANS-3 the first change information item and determining DET-3 the first modified base image are carried out as described above for each of the second medial images. The method steps starting from the transferring TRANS-4 of the first modified base image are therein carried out for every tenth or twentieth or fiftieth or hundredth second medical image. In other words, the method steps of transferring TRANS-4 the first modified base image and the method steps which depend upon the first threshold value and the first similarity value SV_1 are carried out only for every tenth or twentieth or fiftieth or hundredth second medical image. Other arbitrary intervals between the transferred first modified base images are conceivable. For all the other second medical images, the first modified base image is provided directly in the method step of providing PROV-2 the first modified base image. This is represented by the finely dashed arrow.

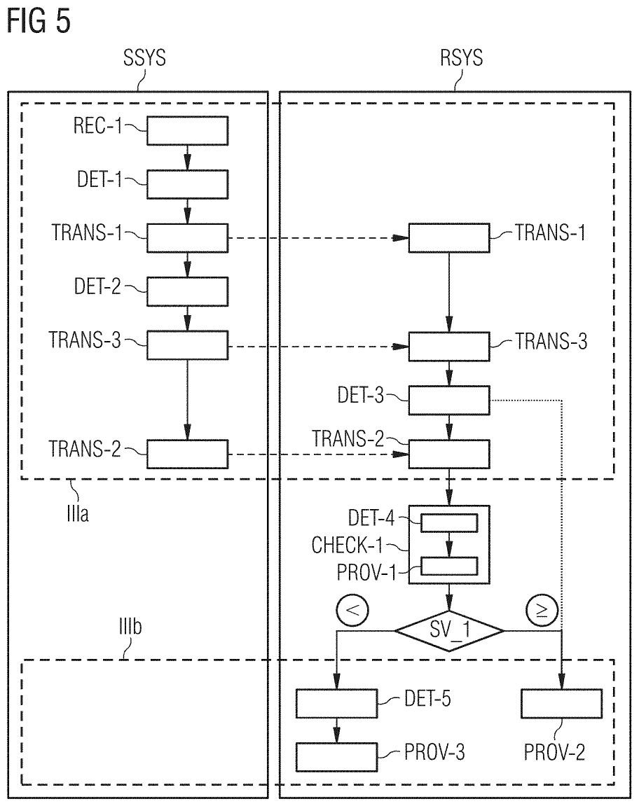

FIG. 5 shows a fifth exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1. The method steps of determining DET-4 the first similarity value SV_1 and providing PROV-1 the first similarity value SV_1 are configured according to the description relating to FIG. 2. The method steps of transferring TRANS-3 the first change information item, providing PROV-2 the first modified base image, determining DET-5 the second base image, transferring TRANS-2′ the second base image and providing PROV-3 the second base image are configured according to the description relating to FIG. 3.

In the exemplary embodiment, the first change information item is transferred independently of the first threshold value and the first similarity value SV_1 in the method step of transferring TRANS-3 the first change information item.

Dependent upon the first base image and the first change information item, in the method step of determining DET-3 the first modified base image, the first modified base image is determined with the receiving apparatus computing unit RSYS.CU.

In the method step of transferring TRANS-2 the second medical image, the second medical image is transferred by the transmitting apparatus SSYS to the receiving apparatus RSYS. In particular, the second medical image is transferred by the transmitting apparatus interface SSYS.IF to the receiving apparatus interface RSYS.IF.

The method steps of determining DET-4 the first similarity value SV_1 and providing PROV-1 the first similarity value SV_1 are carried out by means of the receiving apparatus RSYS. In particular, the method step of determining DET-4 the first similarity value SV_1 with the receiving apparatus computing unit RSYS.CU and the method step of providing PROV-1 the first similarity value SV_1 are carried out with the receiving apparatus interface RSYS.IF.

If the first similarity value is greater than or equal to the first threshold value, the first modified base image is provided by means of the receiving apparatus interface RSYS.IF as described above.

If the first similarity value falls below the first threshold value, in the method step of determining DET-5 the second base image, the second base image is determined on the basis of the second medical image as described above, by means of the receiving apparatus computing unit RSYS.CU. The second base image is provided in the method step of providing PROV-3 the second base image by means of the receiving apparatus interface RSYS.IF as described above.

In embodiments, the plurality of medical images comprises a plurality of second medical images as described above in relation to FIG. 4. Therein, the method steps of receiving REC-1 the plurality of medical images, determining DET-1 the first base image, transferring TRANS-1 the first base image, determining DET-2 the first change information item, transferring TRANS-3 the first change information item and determining DET-3 the first modified base image are carried out as described above for each of the second medial images. The method step of transferring TRANS-2 the second medical image is therein carried out only for every tenth or twentieth or fiftieth or hundredth second medical image. Other arbitrary intervals between the transferred second medical images are conceivable. In particular, the method steps dependent upon the transferring TRANS-2 of the second medical image are then also carried out only for every tenth or twentieth or fiftieth or hundredth second medical image. For all the other second medical images, the first modified base image is provided directly in the method step of providing PROV-2 the first modified base image. This is identified by the finely dashed arrow in the representation.

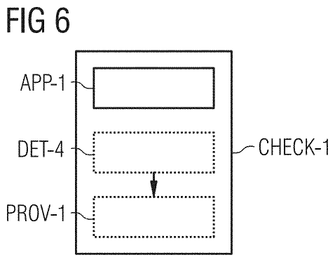

FIG. 6 shows an exemplary embodiment of a method step of checking CHECK-1 a second medical image and/or a first modified base image.

In the exemplary embodiment, the method step of checking CHECK-1 the second medical image and/or the first modified base image comprises a method step of applying APP-1 a second trained function. The second trained function can therein be configured as, in general, similar in relation to the trained function above. The second trained function is therein applied to the first and/or second medical image. Therein, it is determined whether the first and/or the second medical image has an anomaly AN. An anomaly AN can therein describe a deviation from a standard. An anomaly AN can be, in the first and/or second medical image, for example, a hemorrhage and/or a change in a perspective and/or a physiological abnormality of the patient, etc. An anomaly AN is, in particular, a medically relevant detail. The medically relevant detail is described above.

The method step of transferring TRANS-2 the second medical image from the transmitting apparatus interface SSYS.IF to the receiving apparatus interface RSYS.IF is carried out, in particular, if the first and/or the second medical image has an anomaly AN. In particular, the method step of transferring TRANS-2 the second medical image in the form of the transfer TRANS-2′ of the second base image is carried out if the first and/or the second medical image has an anomaly AN.

The second trained function has thereby been trained with a predefined plurality of medical images and/or comprises a long-short-term memory (LSTM) network.

In particular, the second trained function can be based upon a cluster analysis, in particular a k-means-clustering, if the second trained function has been trained with a predefined plurality of second medical images.

Alternatively, the second trained function can comprise an LSTM network. Therein, the second trained function can analyze the temporal sequence of medical images. On the basis of the predefined plurality of medical images, the second trained function can be trained to recognize a standard sequence. Deviations from this standard sequence can be recognized and/or determined as an anomaly AN.

In an embodiment, the predefined plurality of medical images can comprise merely standard images without an anomaly AN. In an alternative embodiment, the predefined plurality of medical images can comprise standard images and images with an anomaly AN.

The second trained function can be trained by means of non-monitored learning on the basis of the predefined plurality of medical images.

Alternatively, the second trained function can be trained by means of monitored learning on the basis of the predefined plurality of medical images. In this case, the predefined plurality of medical images comprises standard images and images with an anomaly AN. Therein, the medical images of the predefined plurality of medical images are annotated. In other words, the medical images of the predefined plurality of medical images are identified as whether they have and/or comprise and/or map and/or represent an anomaly AN or not. The predefined plurality of medical images can be manually annotated for training the second trained function. In other words, the images can be manually identified by an expert, for example a medical practitioner, as to whether they comprise an anomaly AN or not. The second trained function is then trained such that the result corresponds, through the application of the second trained function to a medical image of the predefined plurality of medical images, to the annotation of the corresponding medical image.

In embodiments, the method step of checking CHECK-1 the second medical image and/or the first modified image can comprise the method steps of determining DET-4 and providing PROV-1 the first similarity value SV_1 and/or the method step of applying APP-1 the first trained function. In order to clarify this, the method steps of determining DET-4 and providing PROV-1 the first similarity value SV_1 are shown finely dashed.

The exemplary embodiments according to FIGS. 3 to 4 can similarly be applied to exemplary embodiments in which the method step of checking CHECK-1 the second medical image and/or the first modified base image comprises the method step of applying APP-1 the first trained function. In particular, the method steps which are carried out according to the exemplary embodiments of FIGS. 3 to 5 if the first similarity value SV_1 is greater than or equal to the first threshold value can alternatively be carried out if the first and/or second medical image has no anomaly. The method steps which are carried out according to the exemplary embodiments of FIGS. 3 to 5 if the first similarity value SV_1 falls below the first threshold value can alternatively be carried out if the first and/or second medical image has an anomaly.

In particular, the method step of checking CHECK-1 the second medical image and/or the first modified base image can comprise all three method steps. Then, according to the exemplary embodiments of FIGS. 3 to 5, the method steps that are carried out if the first similarity value SV_1 falls below the first threshold value are carried out if at least the first similarity value SV_1 falls below the first threshold value or if the first and/or second medical image has an anomaly. In other words, the method steps which are carried out according to FIGS. 3 to 5 if the first similarity value SV_1 is greater than or equal to the first threshold value, are only carried out if the first similarity value SV_1 is greater than or equal to the first threshold value and if the first and/or second medical image has no anomaly.

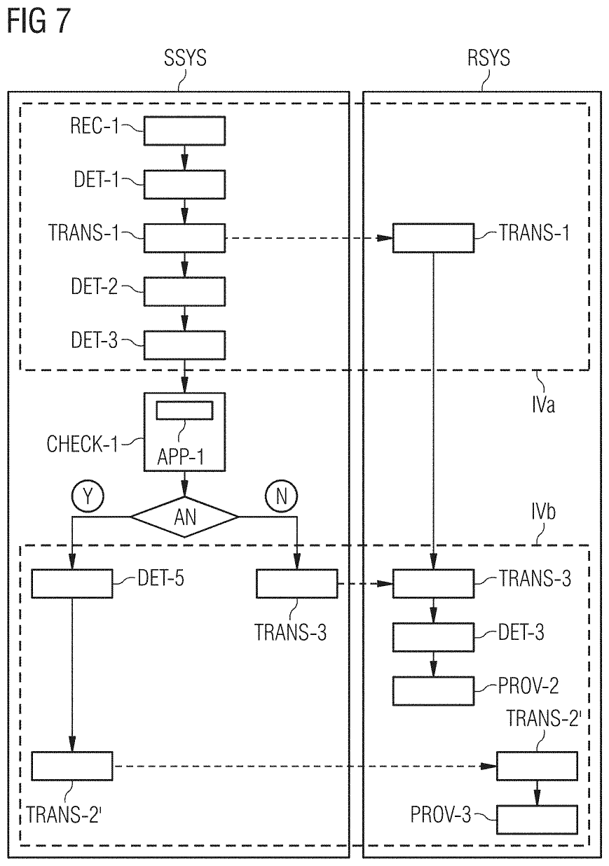

FIG. 7 shows a sixth exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1. The method steps of transferring TRANS-3 the first change information item, providing PROV-2 the first modified base image, determining DET-5 the second base image, transferring TRANS-2′ the second base image and providing PROV-3 the second base image are configured according to the description relating to FIG. 3. The method step of applying APP-1 the second trained function is configured according to the description relating to FIG. 6.

The exemplary embodiment largely corresponds to the third exemplary embodiment according to FIG. 3. As distinct from the third exemplary embodiment, the method step of checking CHECK-1 the second medical image and/or the first modified base image comprises the method step of applying APP-1 the second trained function to the first and/or second medical image. The method steps which are carried out according to the third exemplary embodiment, if the first similarity value SV_1 falls below the first threshold value, are carried out according to the sixth exemplary embodiment if the first and/or second medical image has an anomaly AN. Similarly, the method steps which are carried out according to the third exemplary embodiment if the first similarity value SV_1 is greater than or equal to the first threshold value, are carried out according to the sixth exemplary embodiment if the first and/or second medical image has no anomaly AN.

Similarly, the exemplary embodiments four and five according to FIGS. 4 and 5 can similarly be adapted if the method step of checking CHECK-1 the second medical image and/or the first modified base image comprises the method step of applying APP-1 the second trained function.

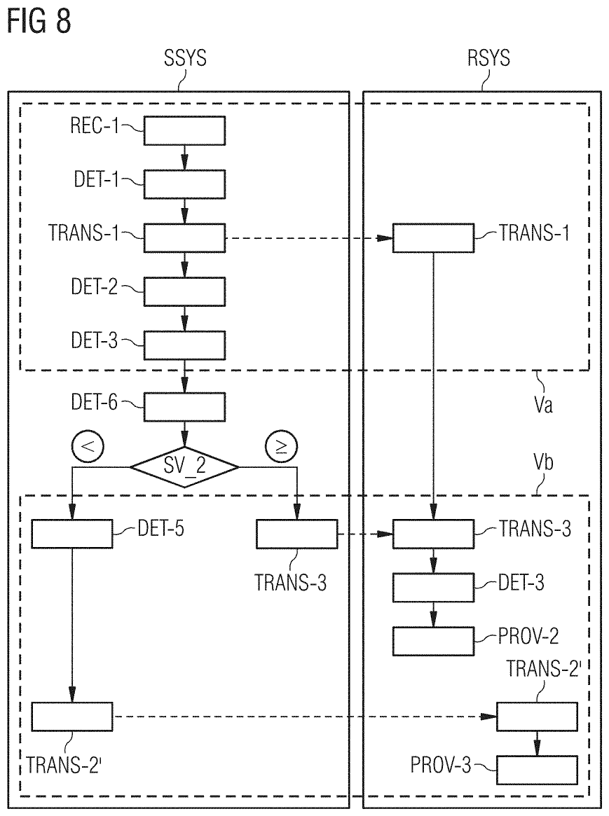

FIG. 8 shows a seventh exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1. The method steps of transferring TRANS-3 the first change information item, providing PROV-2 the first modified base image, determining DET-5 the second base image, transferring TRANS-2′ the second base image and providing PROV-3 the second base image are configured according to the description relating to FIG. 3.

In the method step of determining DET-6 a second similarity value SV_2, the second similarity value SV_2 between the first base image and the second medical image is determined with the transmitting apparatus computing unit SSYS.CU. The second similarity value SV_2 can be determined according to the description relating to FIG. 2 for determining DET-4 the first similarity value SV_1. In other words, the second similarity value SV_2 can depend upon the sum of the squared intervals between the voxel or pixel values of the first base image and the second medical image. Alternatively, the second similarity value SV_2 can be determined by applying a third trained function to the first base image and the second medical image. The third trained function can be configured similarly to the first trained function according to the description relating to FIG. 2.

The second threshold value can be configured similarly to the first threshold value. In the case of a second similarity value SV_2 that is greater than or equal to the second threshold value, it can be assumed that the first base image and the second medical image are similar such that the first modified base image determined dependent upon the first base image and the first change information item comprises all the medically relevant details of the second medical image. If the second similarity value SV_2 falls below the second threshold value, it can be assumed that the first modified base image possibly does not comprise all the medically relevant details of the second medical image.

The seventh exemplary embodiment largely corresponds to the third exemplary embodiment according to FIG. 3. In place of the method step of checking CHECK-1 the second medical image and/or the first modified base image, the method step of determining DET-6 the second similarity value SV_2 is determined. The method steps which are carried out according to FIG. 3 if the first similarity value SV_1 is greater than or equal to the first threshold value, are carried out according to the seventh exemplary embodiment if the second similarity value SV_2 is greater than or equal to the second threshold value. The method steps which are carried out according to FIG. 3 if the first similarity value SV_1 falls below the first threshold value, are carried out according to the seventh exemplary embodiment if the second similarity value SV_2 falls below the second threshold value.

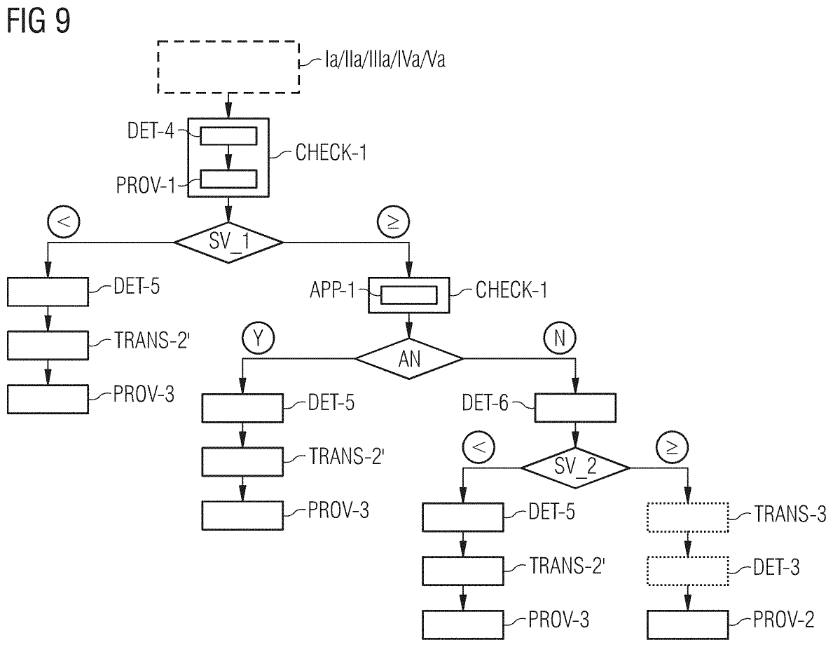

FIG. 9 shows an eighth exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The eighth exemplary embodiment shows schematically a combination of the exemplary embodiments three to seven. In alternative embodiments, the dashed box Ia/IIa/IIIa/IVa/Va describes the method steps included by a correspondingly identified box in FIGS. 3 to 5 and 7 and 8. All the method steps are configured according to the preceding descriptions relating to the figures.

As soon as one of the similarity values SV_1, SV_2 falls below the corresponding threshold value or as soon as an anomaly AN can be determined, the method steps of determining DET-5 the second base image, transferring TRANS-2′ the second base image and providing PROV-3 the second base image are carried out. Therein, the method steps are carried out according to the exemplary embodiment of the transmitting apparatus SSYS and/or the receiving apparatus RSYS. The method steps of transferring TRANS-3 the first change information item, determining DET-3 the first modified base image and providing PROV-2 the first modified base image with the receiving apparatus RSYS are only carried out if the first similarity value is greater than or equal to the first threshold value, the second similarity value is greater than or equal to the second threshold value and if the first and/or second medical image have no anomaly AN. The method steps of transferring TRANS-3 the first change information item and of determining DET-3 the first modified base image with the receiving apparatus RSYS and/or with the receiving apparatus interface RSYS.IF and the receiving apparatus computing unit RSYS.CU are shown dashed, since they are already included by the method steps in the dashed box Ia/IIa/IIIa/IVa/Va, dependent upon the exemplary embodiment.

The order of decisions can be changed arbitrarily. A decision describes a branching point in the method. A decision is identified in the representation in the figures with a diamond shape. In particular also, only two of the three decisions can be carried out. For example, just one decision can be carried out on the basis of the first similarity value SV_1 and the second similarity value SV_2. Alternatively, a decision can be carried out on the basis of the first similarity value SV_1 and the anomaly AN. Alternatively, a decision can be carried out on the basis of the second similarity value SV_2 and the anomaly AN.

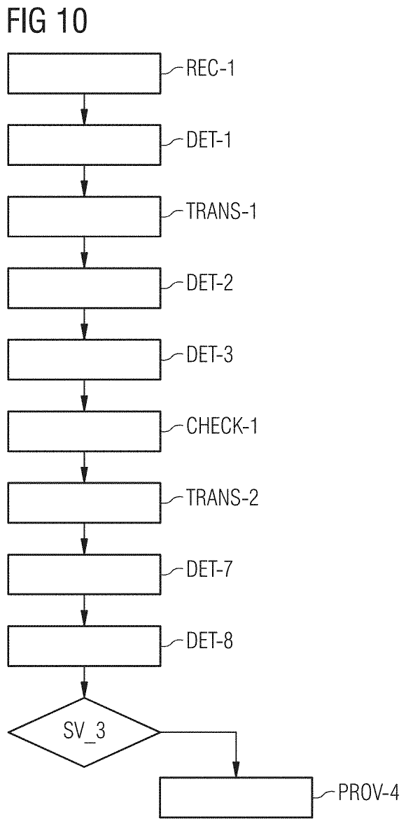

FIG. 10 shows a ninth exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1.

In the exemplary embodiment, the plurality of medical images comprises at least one third medical image. The third medical image is configured similarly to the first and the second medical image. The third medical image has been captured temporally after the second medical image.

In the method step of determining DET-7 a second change information item, the second change information item is determined, dependent upon the first and the third medical images, by means of the transmitting apparatus computing unit SSYS.CU. The second change information item is configured similarly to the first change information item. Whereas the first change information item describes an alteration and/or change between the first medical image and the second medical image, the second change information item describes an alteration and/or change between the first medical image and the third medical image.

In the method step of determining DET-8 a third similarity value SV_3, the third similarity value SV_3 is determined between the first and the second change information item with the transmitting apparatus computing unit SSYS.CU. The third similarity value SV_3 specifies a similarity between the first and the second change information items. The third similarity value can therein be determined, in particular, by applying a fourth trained function to the first and second change information items. The third similarity value SV_3 can specify the similarity, for example, as a percentage similarity. The higher the third similarity value SV_3, the greater is the similarity. Alternatively, the third similarity value SV_3 can specify the similarity of the first and the second change information items in the form of a real number. Therein, the real number can lie between 0 and 1. Therein, 0 can signify that the first and the second change information items are not similar to one another and 1 can signify that the first and the second change information items are identical. Alternatively, the third similarity value SV_3 can classify the similarity. For example, on the basis of the third similarity value SV_3, the similarity between the first and the second change information items can be subdivided into two classes “1” and “0”. In a third similarity value SV_3 of “1”, it can be assumed that the first and second change information items are similar to one another. In particular, the first and second change information items are similar to one another as experience would suggest. With a third similarity value SV_3 of “0”, the first and second change information items are so dissimilar to one another that an error has possibly occurred in the determination DET-2, DET-7 of one of the change information items or the perspective has changed, etc. The perspective of a medical image therein describes a perspective in the medical image onto the portion of the patient being imaged and/or mapped. Alternatively, for example, a sudden occurrence of an anomaly AN, for example, a hemorrhage in the third medical image can have the result that the third similarity value SV_3 is small, in particular, close or equal to 0.

In the method step of providing PROV-4 the third similarity value SV_3, the third similarity value SV_3 is provided with the transmitting apparatus interface SSYS.IF if the third similarity value SV_3 falls below a third threshold value. The third threshold value specifies a minimum similarity which the first and second change information items should have so that it can be assumed that the method and/or the medical intervention proceeds correctly. If the third similarity value SV_3, falls below the third threshold value, the third similarity value SV_3 is provided by means of the transmitting apparatus interface SSYS.IF.

In particular, the third similarity value SV_3 can be provided such that the third similarity value SV_3 is transferred by the transmitting apparatus interface SSYS.IF and the receiving apparatus interface RSYS.IF. Then, the third similarity value SV_3 can be provided to the medical practitioner with the receiving apparatus interface RSYS.IF. In particular, the third similarity value SV_3 can be provided to the medical practitioner by means of the display unit, described above, of the receiving apparatus RSYS. In particular, the third similarity value SV_3 can be provided to the medical practitioner in the form of a set of traffic lights. For example, the traffic lights can switch from “green” to “red” if the third similarity value SV_3 falls below the third threshold value.

The ninth exemplary embodiment can be combined as desired with one of the preceding exemplary embodiments.

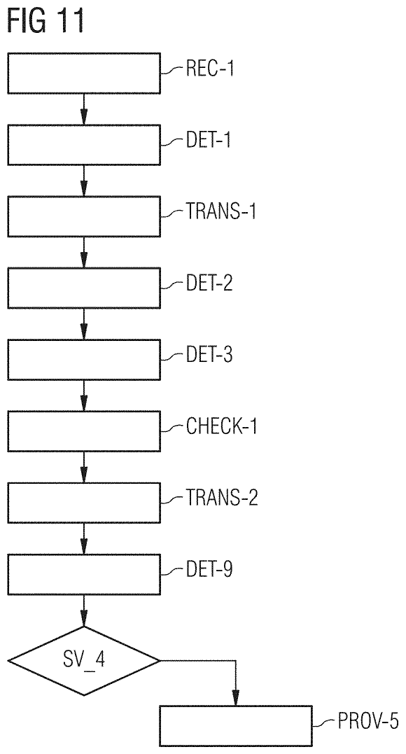

FIG. 11 shows a tenth exemplary embodiment of a computer-implemented method for transferring a plurality of medical images from a transmitting apparatus SSYS comprising a transmitting apparatus interface SSYS.IF and a transmitting apparatus computing unit SSYS.CU to a receiving apparatus RSYS comprising a receiving apparatus interface RSYS.IF and a receiving apparatus computing unit RSYS.CU.

The method steps of receiving REC-1 the plurality of medical images, determining DET-1 the first base image, transferring TRANS-1 the first base image, determining DET-2 the first change information item, determining DET-3 the first modified base image, checking CHECK-1 the second medical image and/or the first modified base image and transferring TRANS-2 the second medical image are configured according to the description relating to FIG. 1.

In the method step of determining DET-9 a fourth similarity value SV_4, by means of the transmitting apparatus computing unit SSYS.CU, the fourth similarity value is determined between the first and second medical images. The fourth similarity value SV_4 can depend, similarly to the above-described first similarity value SV_1 or the second similarity value SV_2, upon the sum of the squared intervals between the voxel or pixel values of the first base image and the second medical images. Alternatively, the fourth similarity value SV_4 can be determined by applying a fifth trained function to the first and the second medical images. The fifth trained function can therein be configured similarly to the first trained function. Alternatively, the fifth trained function can be configured similarly to the second trained function. In particular, the fifth trained function can be based upon a cluster analysis and/or can comprise an LSTM network.

The fourth similarity value SV_4 specifies the similarity between the first and the second medical images. The fourth similarity value SV_4 can specify the similarity as a percentage similarity. The greater the percentage similarity, the more similar the second medical image is to the first medical image. Alternatively, the fourth similarity value SV_4 can specify the similarity as a real number, for example, between 0 and 1. Alternatively, the fourth similarity value SV_4 can specify the similarity of the first and second medical images in the form of a classification. A fourth similarity value SV_4 of “1” can therein specify that the first and the second medical images are as similar to one another as would be expected. A fourth similarity value SV_4 of “0” can specify that the first and the second medical images differ from one another. This can occur, in particular, if one of the two medical images has an anomaly AN and/or a hemorrhage occurs in one of the medical images and/or a perspective on the portion of the patient mapped in the first and/or second medical image changes between the images.

In the method step of providing PROV-5 the fourth similarity value SV_4, the fourth similarity value SV_4 is determined with the transmitting apparatus interface SSYS.IF if a fourth similarity value SV_4 falls below the fourth threshold value. The fourth threshold value specifies a minimum similarity which the first and second change information items should have so that it can be assumed that the medical intervention is proceeding correctly and that the first modified base image can be correctly determined dependent upon the first base image and the first change information item. If the fourth similarity value SV_4 falls below the fourth threshold value, the fourth similarity value SV_4 is provided by means of the transmitting apparatus interface SSYS.IF.

In particular, the fourth similarity value SV_4 can be provided such that the fourth similarity value SV_4 is transferred by the transmitting apparatus interface SSYS.IF and the receiving apparatus interface RSYS.IF. Then, the fourth similarity value SV_4 can be provided to the medical practitioner with the receiving apparatus interface RSYS.IF. In particular, the fourth similarity value SV_4 can be provided to the medical practitioner by means of the display unit, described above, of the receiving apparatus RSYS. In particular, the fourth similarity value SV_4 can be provided in the form of a set of traffic lights. For example, the traffic lights can switch from “green” to “red” if the fourth similarity value SV_4 falls below the fourth threshold value. If the tenth exemplary embodiment is combined with the ninth exemplary embodiment as per FIG. 10, the traffic lights can switch from “green” to “red” if at least the third similarity value SV_3 falls below the third threshold value or if the fourth similarity value SV_4 falls below the fourth threshold value.

The tenth exemplary embodiment can be combined as desired with one of the preceding exemplary embodiments.

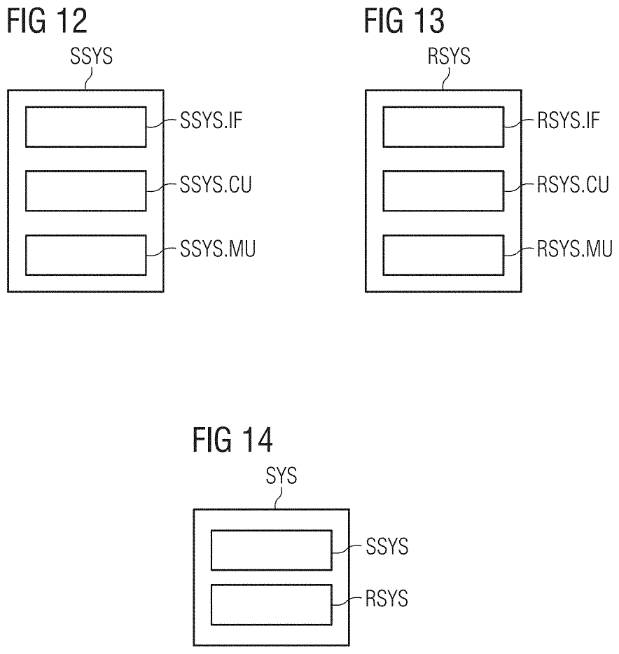

FIG. 12 shows a transmitting apparatus SSYS, FIG. 13 shows a receiving apparatus RSYS and FIG. 14 shows a transfer system SYS for transferring a plurality of medical images.

The transfer system SYS shown for transferring a plurality of medical images is configured to carry out a method according to example embodiments for transferring a plurality of images from a transmitting apparatus SSYS to a receiving apparatus RSYS. The transfer system SYS comprises the transmitting apparatus SSYS and the receiving apparatus RSYS. Therein, the transmitting apparatus SSYS is configured to carry out a transmitting method according to example embodiments. The transmitting method comprises the method steps of the method according to example embodiments for transferring a plurality of medical images, which are carried out according to the description relating to FIGS. 1 to 11 by the transmitting apparatus SSYS. Therein, the receiving apparatus RSYS is configured to carry out a receiving method according to example embodiments. The receiving method comprises the method steps of the method according to example embodiments for transferring a plurality of medical images, which are carried out according to the description relating to FIGS. 1 to 11 by the receiving apparatus RSYS. The transmitting apparatus comprises a transmitting apparatus interface SSYS.IF, a transmitting apparatus computing unit SSYS.CU and a transmitting apparatus memory unit SSYS.MU. The receiving apparatus RSYS comprises a receiving apparatus interface RSYS.IF, a receiving apparatus computing unit RSYS.CU and a receiving apparatus memory unit RSSYS.MU.

The transmitting apparatus SSYS and/or the receiving apparatus RSYS can be, in particular, a computer, a microcontroller or an integrated circuit (IC). Alternatively, the transmitting apparatus SSYS and/or the receiving apparatus RSYS can be a real or virtual computer network (a technical term for a real computer network is a “cluster” and a technical term for a virtual computer network is a “cloud”). The transmitting apparatus SSYS and/or the receiving apparatus RSYS can be configured as a virtual system which is executed on a computer or a real computer network or a virtual computer network (a technical term for this is “virtualization”).

The transmitting apparatus interface SSYS.IF and/or the receiving apparatus interface RSYS.IF can be a hardware or software interface (for example, a PCI bus, a USB or a Firewire). The transmitting apparatus computing unit SSYS.CU and/or the receiving apparatus computing unit RSYS.CU can comprise hardware and/or software constituents, for example, a microprocessor or a so-called field-programmable gate array (FPGA). The transmitting apparatus memory unit SSYS.MU and/or the receiving apparatus memory unit RSYS.MU can be realized as non-permanent working memory (Random Access Memory, (RAM)) or as a permanent mass storage unit (hard disk, USB stick, SD card, solid state disk (SSD)).

The transmitting apparatus interface SSYS.IF and/or the receiving apparatus interface RSYS.IF can, in particular, comprise a plurality of sub-interfaces which carry out different method steps of the respective method according to example embodiments. In other words, the transmitting apparatus interface SSYS.IF and/or the receiving apparatus interface RSYS.IF can be configured as a plurality of transmitting apparatus interfaces SSYS.IF and/or receiving apparatus interfaces RSYS.IF. The transmitting apparatus computing unit SSYS.CU and/or the receiving apparatus computing unit RSYS.CU can comprise, in particular, a plurality of computer sub-units which carry out different method steps of the respective method according to example embodiments. In other words, the transmitting apparatus computing unit SSYS.CU and/or the receiving apparatus computing unit RSYS.CU can be configured as a plurality of computing units SYS.CU and/or training computing units TSYS.CU.

Where it has not yet explicitly been set out, although useful and in the spirit of example embodiments, individual exemplary embodiments, individual sub-aspects or features thereof can be combined and/or exchanged with one another without departing from the scope of example embodiments. Advantages of example embodiments described in relation to an exemplary embodiment also apply, where transferrable, to other exemplary embodiments without this being explicitly stated.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for transferring a plurality of medical images by a transmitting apparatus comprising a transmitting apparatus interface and a transmitting apparatus computing unit to a receiving apparatus comprising a receiving apparatus interface and a receiving apparatus computing unit,
wherein the plurality of medical images comprises a temporal sequence,
wherein the plurality of medical images comprises at least one first medical image and one second medical image,
wherein the first medical image has been captured temporally before the second medical image, the method comprising:
receiving the plurality of medical images with the transmitting apparatus interface;
determining a first base image, dependent upon the first medical image, with the transmitting apparatus computing unit;
transferring the first base image with the transmitting apparatus interface to the receiving apparatus interface;
determining a first change information item, based on the first and second medical images, with the transmitting apparatus computing unit;
determining a first modified base image based on the first change information item and the first base image;
checking at least one of the second medical image or the first modified base image; and
transferring the second medical image with the transmitting apparatus interface to the receiving apparatus interface based on the result of the checking of the at least one of the second medical image or the first modified base image.

2. The method as claimed in claim 1, wherein the checking the at least one of the second medical image or the first modified base image comprises:
determining a first similarity value between the first modified base image and the second medical image; and
providing the first similarity value.

3. The method as claimed in claim 2,
wherein, in the determining the first similarity value, the first similarity value is determined by applying a first trained function to the first modified base image and the second medical image.

4. The method as claimed in claim 2,
wherein the determining the first modified base image,
determining the first similarity value, and providing the first similarity value are carried out by the transmitting apparatus, the method further comprising:
transferring the first change information item with the transmitting apparatus interface to the receiving apparatus interface if the first similarity value is greater than or equal to a first threshold value;
determining the first modified base image with the receiving apparatus computing unit if the first similarity value is greater than or equal to the first threshold value;
providing the first modified base image with the receiving apparatus interface if the first similarity value is greater than or equal to the first threshold value; and
if the first similarity value falls below the first threshold value,
determining a second base image based on the second medical image with the transmitting apparatus computing unit,
transferring the second base image with the transmitting apparatus interface to the receiving apparatus interface, and
providing the second base image with the receiving apparatus interface.

5. The method as claimed in claim 2, further comprising:
transferring the first change information item with the transmitting apparatus interface to the receiving apparatus interface, wherein the determining the first modified base image is carried out by the transmitting apparatus computing unit;
transferring the first modified base image with the receiving apparatus interface to the transmitting apparatus interface,
wherein the determining the first similarity value and the providing the first similarity value are carried out by the transmitting apparatus;
if the first similarity value is greater than or equal to a first threshold value,
providing the first modified base image with the receiving apparatus interface; and
if the first similarity value falls below the first threshold value
determining a second base image based on the second medical image by the transmitting apparatus computing unit,
transferring the second base image by the transmitting apparatus interface to the receiving apparatus interface, and
providing the second base image by the receiving apparatus interface.

6. The method as claimed in claim 2, further comprising:
transferring the first change information item with the transmitting apparatus interface to the receiving apparatus interface, wherein the method step of determining the first modified base image is carried out by the receiving apparatus computing unit;
transferring the second medical image with the transmitting apparatus interface to the receiving apparatus interface,
wherein the method steps of the determining the first similarity value and the providing the first similarity value are carried out by the receiving apparatus;
if the first similarity value is greater than or equal to a first threshold value,
providing the first modified base image with the receiving apparatus interface; and
if the first similarity value falls below the first threshold value,
determining a second base image based on the second medical image with the receiving apparatus computing unit, and
providing the second base image with the receiving apparatus interface.

7. The method as claimed in claim 5,
wherein the plurality of medical images comprises a plurality of second medical images,
wherein the plurality of second medical images have been captured temporally after the first medical image,
wherein the second medical images are arranged in a temporal sequence,
wherein, at least one of (i) the transferring the second medical image with the transmitting apparatus interface to the receiving apparatus interface or (ii) the transferring the first modified image from the receiving apparatus interface to the transmitting apparatus interface and the method steps based on the first threshold value are carried out at predetermined intervals, in particular, for every tenth or every twentieth or every fiftieth second medical image,
wherein for all the other second medical images, the method comprises:
providing the first modified base image with the receiving apparatus interface.

8. The method as claimed in claim 1, wherein the checking at least one of the second medical image or the first modified base image comprises:
applying a second trained function to at least one of the first or the second medical image,
wherein, it is determined whether at least one of the first or second medical image has an anomaly,
wherein the transferring the second medical image from the transmitting apparatus interface to the receiving apparatus interface is carried out if the at least one of the first or the second medical image has an anomaly.

9. The method as claimed in claim 8,
wherein at least one of
(i) the second trained function has been trained with a predefined plurality of medical images, or
(ii) the second trained function comprises a long-short-term memory network.

10. The method as claimed in claim 9, further comprising:
determining a second base image based on the second medical image with the transmitting apparatus computing unit if at least one of the first or second medical image has an anomaly,
transferring the second base image with the transmitting apparatus interface to the receiving apparatus interface if the at least one of the first or second medical image has an anomaly, and
providing the second base image with the receiving apparatus interface if the at least one of the first or second medical image has an anomaly.

11. The method as claimed in claim 1, comprising:
determining a second similarity value between the first base image and the second medical image with the transmitting apparatus computing unit;
if the second similarity value falls below a second threshold value,
determining a second base image based on the second medical image with the transmitting apparatus computing unit,
transferring the second base image by means of the transmitting apparatus interface to the receiving apparatus interface, and
providing the second base image with the receiving apparatus interface; and
if the second similarity value is greater than or equal to the second threshold value,
transferring the first change information item by means of the transmitting apparatus interface to the receiving apparatus interface, wherein the modified first base image is determined with the receiving apparatus interface computing unit, and
providing the modified first base image with the receiving apparatus interface.

12. The method as claimed in claim 11,
wherein in the determining the second similarity value, the second similarity value is determined by applying a third trained function to the first base image and the second medical image.

13. The method as claimed in claim 1,
wherein the plurality of medical images comprises at least one third medical image,
wherein the third medical image has been captured temporally after the second medical image,
wherein the method further comprises:
determining a second change information item upon the first and the third medical image with the transmitting apparatus computing unit;
determining a third similarity value between the first and the second change information item with the transmitting apparatus computing unit; and
providing the third similarity value with the transmitting apparatus interface if the third similarity value falls below a third threshold value.

14. The method as claimed in claim 13,
wherein in the determining the third similarity value, the third similarity value is determined by applying a fourth trained function to the first and second change information items.

15. The method as claimed in claim 1, further comprising:
determining a fourth similarity value between the first medical image and the second medical image with the transmitting apparatus computing unit; and
providing the fourth similarity value with the transmitting apparatus interface if the fourth similarity value falls below a fourth threshold value.

16. The method as claimed in claim 15,
wherein in the determining the fourth similarity value, the fourth similarity value is determined by applying a fifth trained function to the first medical image and the second medical image.

17. A transmitting method comprising:
a transmitting apparatus performing the method of claim 1.

18. A receiving method comprising:
a receiving apparatus performing the method of claim 1.

19. A transmitting apparatus comprising:
a transmitting apparatus interface; and
a transmitting apparatus computing unit,
wherein the transmitting apparatus is configured to carry out the transmitting method as claimed in claim 17.

20. A receiving apparatus comprising:
a receiving apparatus interface; and
a receiving apparatus computing unit,
wherein the receiving apparatus is configured to carry out the receiving method as claimed in claim 18.

21. A transfer system comprising:
a transmitting apparatus; and
a receiving apparatus,
wherein the transmitting apparatus and the receiving apparatus are configured to carry out the method as claimed in claim 1.

22. A computer program product having a computer program which can be loaded directly into a memory of at least one of a transmitting apparatus or a receiving apparatus, having program portions to carry out the method as claimed in claim 1 when the program portions are executed by the at least one of the transmitting apparatus or the receiving apparatus.

23. A computer-readable medium on which program portions that can be read in and executed by at least one of a transmitting apparatus or a receiving apparatus are stored, to carry out the method as claimed claim 1, when the program portions are executed by the at least one of the transmitting apparatus or the receiving apparatus.
